SEQUENCE LISTING (42 sequences)

SEQ ID NO: 1 - DNA, 16 bp, Homo sapiens
cggtggcatcgacaaa

SEQ ID NO: 2 - DNA, 20 bp, Homo sapiens
agcctgagatgtccctgtaa

SEQ ID NO: 3 - DNA, 22 bp, Homo sapiens
accaactgatgtaccagatcaa

SEQ ID NO: 4 - DNA, 23 bp, Homo sapiens
cacctgatgggctctacatataa

SEQ ID NO: 5 - DNA, 18 bp, Homo sapiens
cgacagcgagaagctcaa

SEQ ID NO: 6 - DNA, 19 bp, Homo sapiens
gcctccagctccagaagaa

SEQ ID NO: 7 - DNA, 18 bp, Homo sapiens
ggatctggcactgtggtt

SEQ ID NO: 8 - DNA, 22 bp, Homo sapiens
agaggagcgtctactggtcttt

SEQ ID NO: 9 - DNA, 18 bp, Homo sapiens
agccaagtggaggaagaa

SEQ ID NO: 10 - DNA, 18 bp, Homo sapiens
tagcatgcagtcccacaa

SEQ ID NO: 11 - DNA, 18 bp, Homo sapiens
cccttcagccacatcctt

SEQ ID NO: 12 - DNA, 19 bp, Homo sapiens
ccatctgctttgccacttt

SEQ ID NO: 13 - DNA, 21 bp, Homo sapiens
ctgatttgctgccttatcttt

SEQ ID NO: 14 - DNA, 23 bp, Homo sapiens
gattctggattcagacatctctt

SEQ ID NO: 15 - DNA, 17 bp, Homo sapiens
ggctgagaggcagcgaa

SEQ ID NO: 16 - DNA, 18 bp, Homo sapiens
gctcagggacacctggaa

SEQ ID NO: 17 - DNA, 15 bp, Homo sapiens
aggctccgggctgaa

SEQ ID NO: 18 - DNA, 17 bp, Homo sapiens
cctccaccccactgcaa

SEQ ID NO: 19 - DNA, 22 bp, Homo sapiens
actctctaagccacggaattaa

SEQ ID NO: 20 - DNA, 21 bp, Homo sapiens
ccacaactcctccaatcacaa

SEQ ID NO: 21 - DNA, 19 bp, Homo sapiens
ccatcgctgaaggacagaa

SEQ ID NO: 22 - DNA, 22 bp, Homo sapiens
gctatccacatgagtgagcaaa

SEQ ID NO: 23 - DNA, 18 bp, Homo sapiens
tggcggaccaaagtctaa

SEQ ID NO: 24 - DNA, 18 bp, Homo sapiens
tctctgccctcgtctcaa

SEQ ID NO: 25 - DNA, 22 bp, Homo sapiens
tggctacctctatcaccagatt

SEQ ID NO: 26 - DNA, 22 bp, Homo sapiens
cagcatcagggacttccttatt

SEQ ID NO: 27 - DNA, 21 bp, Homo sapiens
ccagatatcgaggaggtgaaa

SEQ ID NO: 28 - DNA, 21 bp, Homo sapiens
gctgctgagtccatcagagaa

SEQ ID NO: 29 - DNA, 19 bp, Homo sapiens
agcctgatctggtggacaa

SEQ ID NO: 30 - DNA, 20 bp, Homo sapiens
tgtgatgagctcgctggtaa

SEQ ID NO: 31 - DNA, 18 bp, Homo sapiens
gcacagtgccaccacaaa

SEQ ID NO: 32 - DNA, 20 bp, Homo sapiens
cagggagtcgatgaggtcaa

SEQ ID NO: 33 - DNA, 21 bp, Homo sapiens
caaggcctcaagagtcagtaa

SEQ ID NO: 34 - DNA, 18 bp, Homo sapiens
gcgctcgcatctcatcaa

SEQ ID NO: 35 - DNA, 20 bp, Homo sapiens
ggaaagcgaacaaggagtaa

SEQ ID NO: 36 - DNA, 20 bp, Homo sapiens
catcccgtaggtctgtgaaa

SEQ ID NO: 37 - DNA, 19 bp, Homo sapiens
aagtcatggctgctgagaa

SEQ ID NO: 38 - DNA, 19 bp, Homo sapiens
gctgtgatggcgatagcaa

SEQ ID NO: 39 - DNA, 20 bp, Homo sapiens
acgcacacaaaagtgatgaa

SEQ ID NO: 40 - DNA, 20 bp, Homo sapiens
catggtggagtcaggaagaa

SEQ ID NO: 41 - DNA, 22 bp, Homo sapiens
ggctaatgcagagtcaagtaaa

SEQ ID NO: 42 - DNA, 17 bp, Homo sapiens
ggccgtgtatgcctgaa

(19) United States
(12) Patent Application Publication
(10) Pub. No.: US 2005/0282179 A1
(43) Pub. Date: Dec. 22, 2005

(54) Biomarkers for interferon-alpha response in hepatitis C virus infected patients

(75) Inventors: John W. Martin, Pleasant Hill, CA (US); Sheng-Yung P. Chang, Alameda, CA (US)

Correspondence Address: CELERA DIAGNOSTICS, LLC, 1401 HARBOR DAY PARKWAY, ALAMEDA, CA, 94502, US

(73) Assignee: Applera Corporation, Norwalk, CT (US)

(21) Appl. No.: 11/005,881

(22) Filed: Dec. 6, 2004

Related U.S. Application Data
(60) Provisional application No. 60/527,578, filed on Dec. 5, 2003.

(51) Int. Cl. C12Q 1/68; G01N 33/53; C12P 21/06

(52) U.S. Cl. 435/6; 435/69.1; 435/7.1

(57) ABSTRACT

The present invention relates to the identification of prognostic markers useful in determining the response of hepatitis c virus (HCV) infected patients to interferon alpha (IFN-α) treatment. The studies provide biomarkers that can be used to discern sustained responders and non-responders of IFN-α treatment. This information should enable treating physicians to help patients to make more informed decisions.

### FIELD OF THE INVENTION

The present invention relates to the identification of biomarkers useful in determining the response of hepatitis C virus (HCV) infected patients to interferon alpha (IFN-α) treatment. In particular, the invention provides biomarkers that can be used to identify sustained responders and non-responders prior to and/or during IFN-α treatment.

More particularly, the invention relates to a set of marker genes differentially expressed in sustained responder patients (patients with no detectable HCV after a 24 week treatment regime with IFN-α) versus non-responder patients (patients with detectable HCV after a 24 week treatment regime with IFN-α).

### BACKGROUND OF THE INVENTION

HCV infection affects nearly 4 million people in United States and more than 170 million worldwide. Approximately 85% of those infected will develop chronic hepatitis, and up to 20% will progress to cirrhosis in a 10- to 20-year period. Chronic HCV infection is now the most common indication for liver transplantation in the United States (Lauer 2001 N. Engl J. Med 345:41 and Thomas 2000 JAMA 284:450)

IFN-α mono-therapy has been used to treat HCV infection until recently, when the combination of IFN with ribavirin was demonstrated to be significantly more effective. IFN mono-therapy leads to HCV RNA clearance in 30 to 40% of patient during therapy. However, only ˜10 to 15% of these patients has sustained undetectable virus. The IFN and ribavirin combination therapy has proven highly effective, achieving sustained viral eradication in 40% of patients. Recently the FDA approved the combination therapy of pegylated formulation of IFN (PEG-IFN) and ribavirin. The longer half-life of PEG-IFN increases the exposure to the drug, therefore, may increase the efficacy of treatment. Approximately 50% of patients have sustained undetectable levels of HCV after 6-month of PEG-IFN and ribavirin combination therapy. However, the adverse effects of IFN and ribavirin occur in 10 to 20% of patients, and the treatment is usually discontinued in these patients.

Viral factors, such as HCV viral load, and HCV genotype, play a major role in determining the IFN response in HCV chronic infection. Overall, patients with a high viral load and HCV genotype 1, have lower response rate. Other factors, e.g. stage of fibrosis, alcohol assumption, and duration of the infection, also affect the response to IFN treatment. Several host factors, age, sex, and ethnicity, also affect the response to IFN treatment. Predictors of favorable response to the combination therapy include female gender, age of less than 40, and Caucasian race.

Although the detailed mechanisms of anti-viral, anti-proliferative, anti-fibrotic effects of IFN are not clearly understood, the JAK-STAT pathway induced by IFN binding and its receptor on the cell surface has been studied extensively (Stark et al. Ann. Rev. Biochem 67: 227 (1998)). Many genes with IFN-stimulated response elements were activated by transcription factor complex induced by the JAK-STAT pathway. Some of the IFN-inducible proteins, such as 2′, 5′-oligoadenylate synthetase (OAS), double-stranded RNA-dependent protein kinase (PKR), and Mx proteins have well-documented anti-viral activities. Mx protein, a member of GTPase family, is responsible for a specific antiviral state against influenza virus infection in mouse. Several studies correlated the expression level of Mx1 in peripheral mononuclear cells or the mutation in the regulatory region of the Mx1 gene with the response to IFN therapy (Hijikata 2001 Intervirology 44:379 and Meier 2000 J. Med Virol 62:318). In addition, the cellular and humoral immune responses also contribute to persistence of the infection and the development of chronic hepatitis. Liver cell injury and HCV replication may also be immunologically mediated. IFN also has immunomodulatory effect, and it up-regulates the level of HLA class 1 and β-2 microglobulin, and activates macrophages and natural killer cells. Several groups also have identified mutations in immune related genes, e.g. IL-10, TNF-α, and TGF-β and correlated the mutations with response to IFN therapy (Rosen 2002 Am J Gastroenterol 97:714 and Yee 2001 Hepatology 88:708)

Since IFN treatment does not benefit all HCV-infected individuals, and since a significant portion of such individuals exhibit adverse reactions to IFN, there remains a need for a better understanding of the genes involved in the response to IFN treatment and whether they provide useful information in predicting the likelihood of an HCV infected patient's response to treatment.

### SUMMARY OF THE INVENTION

The present invention provides a set of gene markers that distinguish between HCV infected patients that are IFN-α responders from HCV infected patients that are non-responders to IFN-α treatment. In vitro assays were developed for mRNA profiling of 483 genes to investigate the association between the role of host factors and IFN treatment response. The studies provide the opportunities to identify biomarkers that can be used to discern sustained responders and non-responders of IFN treatment. This information should enable treating physicians to make more informed decisions.

The invention further provides a method for assigning a person to one of several categories in a clinical trial, comprising determining for each said person the level of expression of at least 2-15 of the prognosis markers listed in Tables 1, 3, and 4, determining whether the person has an expression pattern that correlates with a responder phenotype or a non-responder phenotype, and assigning said person to one category in a clinical trial if said person is determined to have a responder phenotype, and a different category if that person is determined to have a non-responder phenotype. The invention further provides a method for assigning a person to one of a plurality of categories in a clinical trial, where each of said categories is associated with a different phenotype, comprising determining for each said person the level of expression of at least 2-15 markers from a set of markers, wherein said set of markers includes markers associated with each of said clinical categories, determining therefrom whether the person has an expression pattern that correlates with one of the clinical categories, and assigning said person to one of said categories if said person is determined to have a phenotype associated with that category.

The invention further provides a method of classifying a first cell or organism as having one of at least two different phenotypes, said at least two different phenotypes comprising a first phenotype and a second phenotype, said method comprising: (a) comparing the level of expression of each of a plurality of genes in a first sample from the first cell or organism to the level of expression of each of said genes, respectively, in a pooled sample from a plurality of cells or organisms, said plurality of cells or organisms comprising different cells or organisms exhibiting said at least two different phenotypes, respectively, to produce a first compared value; (b) comparing said first compared value to a second compared value, wherein said second compared value is the product of a method comprising comparing the level of expression of each of said genes in a sample from a cell or organism characterized as having said first phenotype to the level of expression of each of said genes, respectively, in said pooled sample; (c) comparing said first compared value to a third compared value, wherein said third compared value is the product of a method comprising comparing the level of expression of each of said genes in a sample from a cell or organism characterized as having said second phenotype to the level of expression of each of said genes, respectively, in said pooled sample, (d) optionally carrying out one or more times a step of comparing said first compared value to one or more additional compared values, respectively, each additional compared value being the product of a method comprising comparing the level of expression of each of said genes in a sample from a cell or organism characterized as having a phenotype different from said first and second phenotypes but included among said at least two different phenotypes, to the level of expression of each of said genes, respectively, in said pooled sample; and (e) determining to which of said second, third and, if present, one or more additional compared values, said first compared value is most similar, wherein said first cell or organism is determined to have the phenotype of the cell or organism used to produce said compared value most similar to said first compared value.

In a specific embodiment of the above method, said compared values are each ratios of the levels of expression of each of said genes. In another specific embodiment, each of said levels of expression of each of said genes in said pooled sample are normalized prior to any of said comparing steps. In another specific embodiment, normalizing said levels of expression is carried out by dividing each of said levels of expression by the median or mean level of expression of each of said genes or dividing by the mean or median level of expression of one or more housekeeping genes in said pooled sample. In a more specific embodiment, said normalized levels of expression are subjected to a log transform and said comparing steps comprise subtracting said log transform from the log of said levels of expression of each of said genes in said sample from said cell or organism. In another specific embodiment, said at least two different phenotypes are different stages of a disease or disorder. In another specific embodiment, said at least two different phenotypes are different prognoses of a disease or disorder. In yet another specific embodiment, said levels of expression of each of said genes, respectively, in said pooled sample or said levels of expression of each of said genes in a sample from said cell or organism characterized as having said first phenotype, said second phenotype, or said phenotype different from said first and second phenotypes, respectively, are stored on a computer.

The invention provides a kit for determining whether a sample is derived from a patient having an IFN-α responder phenotype or a non-responder phenotype, comprising amplification primers and/or hybridization probes to at least 2-15 of the genes corresponding to the markers listed in Tables 1, 3, and 4. The kit may further comprise a computer readable medium having recorded thereon one or more programs for determining the similarity of the level of nucleic acid derived from the markers listed in Tables 1, 3, and 4 in a sample to that in a pool of samples derived from individuals having a responder phenotype and a pool of samples derived from individuals having a non-responder phenotype, wherein the one or more programs cause a computer to perform a method comprising computing the aggregate differences in expression of each marker between the sample and the responder phenotype pool and the aggregate differences in expression of each marker between the sample and the non-responder phenotype pool, or a method comprising determining the correlation of expression of the markers in the sample to the expression in the responder phenotype and non-responder phenotype pools.

### BRIEF DESCRIPTION OF THE FIGURES

FIG. 1 presents a summary of prognostic factors used in assessing whether an HCV infected patient will respond to IFN-α treatment.

FIG. 2 presents the characteristics of the patients from whom samples were obtained.

FIG. 3 presents a summary diagram of the sample treatment protocol.

FIG. 4 presents the results of univariable analyses of the differential expression of specific markers associated with a response of IFN-α treatment of the samples. Markers above the dotted line survived the conservative Bonferroni Correction.

FIG. 5 presents an unsupervised clustering of the patients based on the expression levels of specific genes identified prior to in vitro IFN-α treatment. In panel A, the patients were clustered based on the expression levels of 5 genes analyzed from Table 4. SVR stands for sustained responders, those patients that responded to treatment. Also included is the distribution of “healthy” individuals who are HCV free. Panel B presents the clustering of the patients in the study based on the expression level of 10 genes from Table 4. The normalized expression level of each transcript of interest was determined for each sample and averaged across all of the samples and the color scale represents the log difference in expression of each transcript of interest in each sample relative to the average expression across all samples.

FIG. 6 presents an unsupervised clustering of patients based on the expression level of 10 specific genes presented in Table 4 and FIG. 5B. The red dots indicate those patients that were sustained responders to IFN-α treatment. Panel A presents the patient clustering based on the expression levels of the 10 indicated genes prior to in vitro IFN-α treatment. Panel B presents the clustering of patients based on the expression levels of the indicated genes after 6 hours of in vitro IFN-α treatment. RNA concentrations for each transcript were determined as described in FIG. 5. After 6 hours of in vitro treatment, the indicated genes were dramatically over expressed in the sustained responders relative to the expression levels prior to treatment.

### DEFINITIONS

The terms “isolated,” “purified,” or “biologically pure” refer to material that is substantially or essentially free from components that normally accompany it as found in its native state. Purity and homogeneity are typically determined using analytical chemistry techniques such as polyacrylamide gel electrophoresis or high performance liquid chromatography. A protein or nucleic acid that is the predominant species present in a preparation is substantially purified. The term “purified” denoted that a nucleic acid or protein gives rise to essentially one band in an electrophoretic gel. Particularly, it means that the nucleic acid or protein is at least 85% pure, more preferably at least 95% pure, and most preferably at least 99% pure.

“Nucleic acid” refers to deoxyribonucleotides, or ribonucleotides and polymers thereof in either single- or double-stranded form. The term encompasses nucleic acids containing known nucleotide analogs or modified backbone residues or linkages, which are synthetic, naturally occurring and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

“Responder phenotype” refers to the phenotype of an HCV infected patient that responds to the normal course of IFN-α treatment such that at the end of the 24-week treatment period the patient does not have any detectable HCV.

“Non-responder phenotype” refers to the phenotype of an HCV infected patient that does not respond to the normal course of IFN-α treatment such that at the end of the 24-week treatment period, the patient has detectable virus.

“Marker” means an entire gene or portion thereof, or an EST derived from that gene, the expression level of which changes between certain conditions. Where the expression of the gene correlates with a certain condition, the gene is a marker for that condition.

“Marker-derived polynucleotides” means the RNA transcribed from a marker gene, any cDNA or mRNA produced therefrom, and any nucleic acid derived therefrom, such as synthetic nucleic acid having a sequence derived from the gene corresponding to the marker gene.

“Altered expression” means that the expression of a marker in an IFN treated sample may be increased or decreased relative to the expression of that marker in an untreated sample.

### DETAILED DESCRIPTION OF THE INVENTION

The invention relates to sets of genetic markers whose expression patterns correlate with important characteristics of response to interferon treatment of individuals infected with HCV. More specifically, the invention provides for sets of genetic markers that can distinguish between patients that respond to IFN-α therapy (Sustained Viral Responders or SVR) and patients that do not respond to IFN-α therapy (Non-Responders or NR). Methods are provided for use of these markers to distinguish between these patient groups, and to determine general courses of treatment. Microarrays comprising these markers are also provided, as well as methods of constructing such microarrays. In a preferred embodiment, kinetic RT-PCR can be used to perform gene expression profiling on the markers of interest. Each marker corresponds to a gene in the human genome, i.e., such marker is identifiable as all or a portion of a gene. Because each of the markers described herein correlates with a certain IFN response-related conditions, the markers, or the proteins they encode, are likely to be targets for drugs to enhance IFN response.

Markers Useful in Determining IFN-α Response in HCV Infected Patients

Marker Sets

The invention provides a set of genetic markers whose expression is correlated with a responder or non-responder phenotype. Clustering analysis can distinguish between patients with a responder phenotype and a non-responder phenotype. These markers are identified in Table 1. A subset of these markers are also identified as useful for prognosis are listed in Table 3.

Table 1. Markers useful for determining IFN responsiveness, i.e. responder and non-responder phenotypes. Columns 1, 3, and 5 provide the Locus Link accession number for each marker gene. Columns 2, 4, and 6 provide the gene symbol for each of the respective marker genes listed in columns 1, 3, and 5. Locus Link is a web site maintained by the National Center for Biotechnology Information (NCBI). Locus Link provides information about the marker gene and its encoded protein of interest including links the GenBank accession numbers that provide both the nucleic acid and amino acid sequences of the marker gene and encoded protein. Based on this information, one of skill in the art would be able to devise and construct primers and probes to analyze the expression of each marker gene of interest in a sample obtained from a patient in accordance with the invention described herein.

| | | Locus | | Locus | |
|---|---|---|---|---|---|
| Locus Link_ID | LL_SYMBOL | Link_ID | LL_SYMBOL | Link_ID | LL_SYMBOL |
| 6059 | ABCE1 | 2669 | GEM | 4939 | OAS2 |
| 9619 | ABCG1 | 2766 | GMPR | 4940 | OAS3 |
| 25 | ABL1 | 2770 | GNAI1 | 8638 | OASL |
| 47 | ACLY | 2782 | GNB1 | 5029 | P2RY2 |
| 91703 | ACY-3 | 2876 | GPX1 | 10135 | PBEF |
| 4185 | ADAM11 | 2885 | GRB2 | 8850 | PCAF |
| 8754 | ADAM9 | 51079 | GRIM19 | 5106 | PCK2 |
| 103 | ADAR | 2896 | GRN | 5110 | PCMT1 |
| 108 | ADCY2 | 2954 | GSTZ1 | 5121 | PCP4 |
| 11047 | ADRM1 | 2962 | GTF2F1 | 5154 | PDGFA |
| 166 | AES | 9567 | GTPBP | 5155 | PDGFB |
| 284602 | AGRN | 10562 | GW112 | 56034 | PDGFC |
| 199 | AIF1 | 2998 | GYS2 | 5157 | PDGFRL |
| 9447 | AIM2 | 3005 | H1F0 | 8566 | PDXK |
| 231 | AKR1B1 | 3028 | HADH2 | 5196 | PF4 |
| 214 | ALCAM | 54363 | HAO1 | 22822 | PHLDA1 |
| 10947 | AP3M2 | 3046 | ABE1 | 5286 | PIK3C2A |
| 338 | APOB | 3066 | HDAC2 | 5287 | PIK3C2B |
| 347 | APOD | 3082 | HGF | 5289 | PIK3C3 |
| 369 | ARAF1 | 3091 | HIF1A | 5292 | PIM1 |
| 389 | ARHC | 8334 | HIST1H2AC | 9600 | PITPNM1 |
| 29984 | ARHD | 8370 | HIST2H4 | 5321 | PLA2G4A |
| 396 | ARHGDIA | 3105 | HLA-A | 5322 | PLA2G5 |
| 9138 | ARHGEF1 | 3106 | HLA-B | 5329 | PLAUR |
| 25820 | ARIH1 | 3107 | HLA-C | 5340 | PLG |
| 440 | ASNS | 3133 | HLA-E | 5352 | PLOD2 |
| 483 | ATP1B3 | 8091 | HMGA2 | 5359 | PLSCR1 |
| 533 | ATP6V0B | 3161 | HMMR | 5366 | PMAIP1 |
| 558 | AXL | 3309 | HSPA5 | 5371 | PML |
| 567 | B2M | 3339 | HSPG2 | 9512 | PMPCB |
| 573 | BAG1 | 3383 | ICAM1 | 5393 | PMSCL1 |
| 578 | BAK1 | 3394 | ICSBP1 | 10585 | PMOT1 |
| 596 | ACL2 | 3397 | ID1 | 5444 | PON1 |
| 635 | BHMT | 3399 | ID3 | 5445 | PON2 |
| 6046 | BRD2 | 3418 | IDH2 | 5480 | PPIC |
| 55290 | BRF2 | 8870 | IER3 | 5504 | PPP1R2 |
| 684 | BST2 | 3428 | IFI16 | 5530 | PPP3CA |
| 694 | BTG1 | 3429 | IFI27 | 5536 | PPP5C |
| 695 | BTK | 10437 | IFI30 | 23532 | PRAME |
| 706 | BZRP | 3430 | IFI35 | 639 | PRDM1 |
| 22918 | C1QR1 | 3427 | IFI4 | 5586 | PRKCL2 |
| 715 | C1R | 10561 | IFI44 | 5610 | PRKR |
| 716 | C1S | 3434 | IFIT1 | 8575 | PRKRA |
| 717 | C2 | 3433 | IFIT2 | 5612 | PRKRIR |
| 83747 | C20ORF57 | 8376 | IFIT3 | 5663 | PSEN1 |
| 757 | C21ORF4 | 3437 | IFIT4 | 5669 | PSG1 |
| 719 | C3AR1 | 8519 | IFITM1 | 5683 | PSMA2 |
| 722 | C4BPA | 10581 | IFITM2 | 5699 | PSMB10 |
| 819 | CAMLG | 10410 | IFITM3 | 5696 | PSMB8 |
| 824 | CAPN2 | 3439 | IFNA1 | 5698 | PSMB9 |
| 834 | CASP1 | 3454 | IFNAR1 | 5714 | PSMD8 |
| 835 | CASP2 | 3455 | IFNAR2 | 5720 | PSME1 |
| 836 | CASP3 | 3456 | IFNB1 | 5721 | PSME2 |
| 837 | CASP4 | 3458 | IFNGR1 | 10197 | PSME3 |
| 841 | CASP8 | 3459 | IFNGR2 | 80142 | PTGES2 |
| 865 | CBFB | 3460 | IFRD2 | 5742 | PTGS1 |
| 885 | CCK | 7866 | IFRG28 | 5743 | PTGS2 |
| 6363 | CCL19 | 64108 | IGF1 | 5770 | PTPN1 |
| 6347 | CCL2 | 3479 | IGF2 | 5894 | RAF1 |
| 6348 | CCL3 | 3481 | IGHM | 5901 | RAN |
| 6351 | CCL4 | 3507 | IGLL3 | 5902 | RANBP1 |
| 6352 | CCL5 | 3545 | IL10 | 5925 | RB1 |
| 896 | CCND3 | 3586 | IL12A | 5928 | RBBP4 |
| 1230 | CCR1 | 3592 | IL12B | 5931 | RBBP7 |
| 10332 | CD209L | 3593 | IL13 | 5937 | RBMS1 |
| 961 | CD47 | 3596 | IL15 | 51109 | RDH11 |
| 965 | CD58 | 3600 | IL15RA | 5965 | RECQL |
| 972 | CD74 | 3601 | IL18 | 5970 | RELA |
| 975 | CD81 | 3606 | IL1A | 5981 | RFC1 |
| 993 | CDC25A | 3552 | IL1B | 24138 | RI58 |
| 1003 | CDH5 | 3553 | IL2 | 6041 | RNASEL |
| 1052 | CEBPD | 3558 | IL3 | 6147 | RPL23A |
| 1147 | CHUK | 3562 | IL4 | 6232 | RPS27 |
| 1152 | CKB | 3565 | IL5 | 6235 | RPS29 |
| 1185 | CLCN6 | 3567 | IL6 | 6303 | SAT |
| 7122 | CLDN5 | 3569 | IL7 | 6383 | SDC2 |
| 1192 | CLIC1 | 3576 | IL8 | 6386 | SDCBP |
| 7373 | COL14A1 | 3620 | INDO | 27111 | SDCBP2 |
| 1307 | COL16A1 | 3624 | INHBA | 6402 | SELL |
| 80781 | COL18A1 | 8826 | IQGAP1 | 5054 | SERPINE1 |
| 1277 | COL1A1 | 3659 | IRF1 | 710 | SERPLNG1 |
| 1278 | COL1A2 | 3660 | IRF2 | 10291 | SF3A1 |
| 1281 | COL3A1 | 3661 | IRF3 | 10946 | SF3A3 |
| 1291 | COL6A1 | 3662 | IRF4 | 10262 | SF3B4 |
| 1292 | COL6A2 | 3663 | IRF5 | 6421 | SFPQ |
| 1293 | COL6A3 | 3664 | IRF6 | 6435 | SFTPA1 |
| 9276 | COPB2 | 3665 | IRF7 | 6440 | SFTPC |
| 10063 | COX17 | 3667 | IRS1 | 6464 | SHC1 |
| 1387 | CREBBP | 3669 | ISG20 | 7979 | SHFM1 |
| 1428 | CRYM | 10379 | ISGF3G | 6472 | SHMT2 |
| 1453 | CSNK1D | 3673 | ITGA2 | 6500 | SKP1A |
| 8048 | CSRP3 | 3716 | JAK1 | 7884 | SLBP |
| 1490 | CTGF | 3717 | JAK2 | 6574 | SLC20A1 |
| 1493 | CTLA4 | 3725 | JUN | 6520 | SLC3A2 |
| 1509 | CTSD | 3730 | KAL1 | 8140 | SLC7A5 |
| 3627 | CXCL10 | 10945 | KDELR1 | 8435 | SOAT2 |
| 6373 | CXCL11 | 23185 | KIAA0217 | 8651 | SOCS1 |
| 4283 | CXCL9 | 3959 | LGALS3BP | 6667 | SP1 |
| 2833 | CXCR3 | 3980 | LIG3 | 3431 | SP110 |
| 1537 | CYC1 | 3988 | LIPA | 6678 | SPARC |
| 54205 | CYCS | 3998 | LMAN1 | 22928 | SPS2 |
| 3491 | CYR61 | 4038 | LRP4 | 6713 | SQLE |
| 1611 | DAP | 4053 | LTBP2 | 6720 | SREBF1 |
| 7818 | DAP3 | 4061 | LY6E | 6737 | SSA1 |
| 1612 | DAPK1 | 4067 | LYN | 6738 | SSA2 |
| 780 | DDR1 | 8379 | MAD1L1 | 6742 | SSBP1 |
| 4921 | DDR2 | 4089 | MADH4 | 6745 | SSR1 |
| 1662 | DDX10 | 5604 | MAP2K1 | 10735 | STAG2 |
| 10521 | DDX17 | 4294 | MAP3K10 | 6770 | STAR |
| 1654 | DDX3X | 9020 | MAP3K14 | 90627 | STARD13 |
| 1660 | DHX9 | 1326 | MAP3K8 | 6772 | STAT1 |
| 9228 | DLGAP2 | 5594 | MAPK1 | 6773 | STAT2 |
| 3300 | DNAJB2 | 5595 | MAPK3 | 6774 | STAT3 |
| 5611 | DNAJC3 | 5597 | MAPK6 | 6775 | STAT4 |
| 10589 | DRAP1 | 5648 | MASP1 | 6776 | STAT5A |
| 1820 | DRIL1 | 10747 | MASP2 | 6777 | STAT5B |
| 1828 | DSG1 | 4150 | MAZ | 6778 | STAT6 |
| 1839 | DTR | 4153 | MBL2 | 10630 | T1A-2 |
| 1890 | ECGF1 | 4175 | MCM6 | 6890 | TAP1 |
| 1948 | EFNB2 | 4218 | MEL | 6891 | TAP2 |
| 1958 | EGR1 | 4261 | MHC2TA | 6897 | TARS |
| 1959 | EGR2 | 4288 | MKI67 | 11138 | TBCID8 |
| 1962 | EHHADH | 4312 | MMP1 | 7004 | TEAD4 |
| 1967 | EIF2B1 | 4323 | MMP14 | 7006 | TEC |
| 1965 | EIF2S1 | 4324 | MMP15 | 7035 | TFPI |
| 8894 | EIF2S2 | 4325 | MMP16 | 7039 | TGFA |
| 8661 | EIF3S10 | 4326 | MMP17 | 7040 | TGFB1 |
| 3646 | EIF3S6 | 4313 | MMP2 | 7042 | TGFB2 |
| 1977 | EIF4E | 10893 | MMP24 | 7043 | TGFB3 |
| 1982 | EIF4G2 | 64386 | MMP25 | 7056 | THBD |
| 2005 | ELK4 | 4314 | MMP3 | 7057 | THBS1 |
| 2023 | ENO1 | 4318 | MMP9 | 7076 | TIMP1 |
| 2033 | EP300 | 4330 | MN1 | 7077 | TIMP2 |
| 2058 | EPRS | 4332 | MNDA | 51284 | TLR7 |
| 2060 | EPS15 | 4353 | MPO | 7190 | TMEM1 |
| 2069 | EREG | 4360 | MRC1 | 7114 | TMSB4X |
| 2114 | ETS2 | 10865 | MRF-1 | 7124 | TNF |
| 2130 | EWSR1 | 4485 | MST1 | 8797 | TNFRSF10A |
| 2173 | FABP7 | 4490 | MT1B | 8795 | TNFRSF10B |
| 2192 | FBLN1 | 9961 | MVP | 355 | TNFRSF6 |
| 2199 | FBLN2 | 4599 | MX1 | 8743 | TNFSF10 |
| 2207 | FCER1G | 4600 | MX2 | 64222 | TOR3A |
| 2246 | FGF1 | 4609 | MYC | 7168 | TPM1 |
| 2247 | FGF2 | 4615 | MYD88 | 8717 | TRADD |
| 2266 | FGG | 9172 | MYOM2 | 9830 | TRIM14 |
| 2281 | FFKBP1B | 4661 | MYT1 | 10346 | TRIM22 |
| 10468 | FST | 10276 | NET1 | 7726 | TRIM26 |
| 8880 | FUBP1 | 4778 | NFE2 | 53840 | TRIM34 |
| 2524 | FUT2 | 4783 | NFIL3 | 7295 | TXN |
| 2526 | FUT4 | 4790 | NFKB1 | 7318 | UBE1L |
| 2534 | FYN | 4791 | NFKB2 | 7341 | UBL1 |
| 9636 | G1P2 | 4792 | NFKBIA | 7351 | UCP2 |
| 2537 | G1P3 | 4803 | NGFB | 7384 | UQCRC1 |
| 2547 | G22P1 | 22795 | NID2 | 7375 | USP4 |
| 2539 | G6PD | 9111 | NMI | 10493 | VAT1 |
| 1647 | GADD45A | 4843 | NOS2A | 7409 | VAV1 |
| 2633 | GBP1 | 4883 | NPR3 | 7422 | VEGF |
| 2634 | GBP2 | 8013 | NR4A3 | 7424 | VEGFC |
| 25801 | GCA | 51667 | NYREN18 | 7448 | VTN |
| 2643 | GCH1 | 4938 | OAS1 | 7453 | WARS |
| 8565 | YARS | 7531 | YWHAE | | |

Table 2. The locus link information for the housekeeping genes used to determine the relative amount of expression of the marker genes presented in Tables 1, 3, and 4 are presented. Also presented are the primers used to amplify each housekeeping gene in an RT-PCR reaction. The column labeled U_SEQ presents the upper primer used to amplify the housekeeping gene and the column labeled L_SEQ presents the lower primer used to amplify the housekeeping gene in the normalization assays.

TABLE 2

Housekeeping genes used in normalization assay.

| LOCUS_{—} | | | |
|---|---|---|---|
| ID | LL_Symbol | U_SEQ (5′-3′) | L_SEQ (5′-3′) |
| 1915 | EEF1A1.1 | CGGTGGCATCGACAAA | AGCCTGAGATGTCCCTGTAA |
| | | (SEQ ID NO: 1) | (SEQ ID NO: 2) |
| 5501 | PPP1CC.1 | ACCAACTGATGTACCAGATCAA | CACCTGATGGGCTCTACATATAA |
| | | (SEQ ID NO: 3) | (SEQ ID NO: 4) |
| 5499 | PPP1CA.1 | CGACAGCGAGAAGCTCAA | GCCTCCAGCTCCAGAAGAA |
| | | (SEQ ID NO: 5) | (SEQ ID NO: 6) |
| 6168 | RPL37A.1 | GGATCTGGCACTGTGGTT | AGAGGAGCGTCTACTGGTCTTT |
| | | (SEQ ID NO: 7) | (SEQ ID NO: 8) |
| 6171 | RPL41.1 | AGCCAAGTGGAGGAAGAA | TAGCATGCAGTCCCACAA |
| | | (SEQ ID NO: 9) | (SEQ ID NO: 10) |

A subset of the markers of Table 1 was identified and is presented in Table 3. These markers are also useful for determining IFN prognosis and responder and non-responder phenotypes. Columns 1, 3 and 5 provide the Locus Link accession number for each marker identified. Columns 2, 4 and 6 provide the locus link gene symbol. After the gene symbol, there is a “.” followed by a number. The number indicates how many splice variants may be amplified in an RT-PCR reaction using a set of primers (see below).

TABLE 3
108 Gene subset list of Table 1.

| LOCUS_ID | LL_Symbol | LOCUS_ID | LL_Symbol | LOCUS_ID | LL_Symbol |
|---|---|---|---|---|---|
| 103 | ADAR.1, 2, 3, 4, 5 | 8519 | IFTIM1.1 | 5610 | PRKR.1 |
| 9447 | AIM2.1 | 10581 | IFITM2.1 | 8575 | PRKRA.1 |
| 567 | B2M.1 | 3454 | IFNAR1.1 | 5612 | PRKRIR.1 |
| 596 | BCL2.1 | 3455 | IFNAR2.1, 2, 3 | 5696 | PSMB8.1, 2 |
| 834 | CASP1.1, 2, 3, 4, 5 | 3455 | IFNAR2.2 | 5696 | PSMB9.1, 2 |
| 835 | CASP2.1, 2, 3, 4 | 3600 | IL15.1, 2, 3 | 5721 | PSME2.1 |
| 836 | CASP3.1, 2, 3 | 3601 | IL15RA.1, 2 | 24138 | RI58.1 |
| 837 | CASP4.1, 2, 3 | 3659 | IRF1.1 | 6041 | RNASEL.1 |
| 841 | CASP8.1, 2, 3, 4, 5 | 3660 | IRF2.1 | 6232 | RPS27.1 |
| 6347 | CCL2.1 | 3661 | IFR3.1 | 6303 | SAT.1, 2 |
| 6351 | CCL4.1 | 3662 | IRF4.1, 2 | 5054 | SERPINE1.1 |
| 965 | CD58.1, 2 | 3663 | IRF5.1, 2 | 8651 | SOCS1.1 |
| 972 | CD74.1 | 3664 | IRF6.1 | 3431 | SP110.1, 2, 3 |
| 975 | CD81.1 | 3665 | IRF7.1, 2, 3, 4 | 6737 | SSA1.1 |
| 1052 | CEBPD.1 | 3667 | IRS1.1 | 6772 | STAT1.2 |
| 1493 | CTLA4.1 | 3669 | ISG20.1 | 6772 | STAT1.1, 2 |
| 3627 | CXCL10.1 | 10379 | ISFG3G.1 | 6773 | STAT2.1 |
| 6373 | CXCL11.1 | 3716 | JAK1.1 | 6774 | STAT3.1, 2 |
| 4283 | CXCL9.1 | 3717 | JAK2.1 | 6775 | STAT4.1 |
| 10521 | DDX17.1, 2, 3 | 3988 | LIPA.1 | 6776 | STAT5A.1 |
| 5611 | DNAJC3.1 | 1326 | MAP3K8.1, 2 | 6777 | STAT5B.1 |
| 1967 | EIF2B1.1 | 4323 | MMP14.1 | 6778 | STAT6.1 |
| 9636 | G1P2.1 | 4332 | MNDA.1 | 6890 | TAP1.1 |
| 2537 | G1P3.1, 2, 3 | 4599 | MX1.1 | 6897 | TARS.1, 2 |
| 2633 | GBP1.1 | 4600 | MX2.1 | 11138 | TBC1D8.1 |
| 2634 | GBP2.1 | 4615 | MYD88.1 | 51284 | TLR7.1 |
| 3383 | ICAM1.1 | 9111 | NMI.1 | 8797 | TNFRSF10A.1 |
| 3394 | ICSPB1.1 | 8013 | NR4A3.1, 2, 3, 4, 5 | 8795 | TNFRSF10B.1, 2 |
| 3428 | IFI16.1 | 51667 | NYREN18.1 | 8743 | TNFSF10.1 |
| 3429 | IFI27.1 | 4938 | OAS1.1, 2 | 8717 | TRADD.1, 2 |
| 10437 | IFI30.1 | 4939 | OAS2.1, 2 | 9830 | TRIM14.1, 2, 3, 4 |
| 3430 | IFI35.1 | 4940 | OAS3.1 | 10346 | TRIM22.1 |
| 10561 | IFI44.1 | 8638 | OASL.1 | 7726 | TRIM26.1 |
| 3434 | IFIT1.1 | 5329 | PLAUR.1, 2, 3 | 53840 | TRIM34.1, |
| | | | | | 2, 3, 4, 5 |
| 3433 | IFIT2.1 | 5359 | PLSCR1.1 | 7453 | WARS.1 |
| 3437 | IFIT4.1, 2 | 5371 | PML.1, 2, 3, 4, 5, 6, | 8565 | YARS.1 |
| | | | 7, 8, 9, 10, 11, 12 | | |

A subset of the markers of Tables 1 and 3 were identified and presented in Table 4. These markers are also useful for determining IFN prognosis and responder and non-responder phenotypes. Locus link and primer information for each of these markers may be found in Table 4. As above, after the gene symbol, there is a “.” followed by a number. The number indicates how many splice variants may be amplified in an RT-PCR reaction using a set of primers in columns 3 and 4. For example, the primers identified in row 1 will amplify 5 alternatively spliced transcripts from the ADAR gene marker (ADAR. 1, 2, 3, 4, 5). The column labeled U_SEQ presents the upper primer used to amplify the indicated marker sequence and the column labeled L_SEQ presents the lower primer used to amplify the indicated marker sequence in assays designed to determine the expression levels of the indicated markers. Based on this information, one of skill in the art would be able to devise and construct additional primers and probes to analyze the expression of each marker gene of interest in a sample obtained from a patient.

TABLE 4

A subset of markers from Table 3.

| | | | |
|---|---|---|---|
| LOCUS | | | |
| _ID | LL_Symbol | U_SEQ (5′-3′) | L_SEQ (5′-3′) |

| | | | |
|---|---|---|---|
| 103 | ADAR.1,2,3,4,5 | CCCTTCAGCCACATCCTT | CCATCTGCTTTGCCACTTT |
| | | (SEQ ID NO: 11) | (SEQ ID NO: 12) |
| 3627 | CXCL10.1 | CTGATTTGCTGCCTTATCTTT | GATTCTGGATTCAGACATCTCTT |
| | | (SEQ ID NO: 13) | (SEQ ID NO: 14) |
| 9636 | G1P2.1 | GGCTGAGAGGCAGCGAA | GCTCAGGGACACCTGGAA |
| | | (SEQ ID NO: 15) | (SEQ ID NO: 16) |
| 2537 | G1P3.1,2,3 | AGGCTCCGGGCTGAA | CCTCCACCGCACTGCAA |
| | | (SEQ ID NO: 17) | (SEQ ID NO: 16) |
| 3429 | IFI27.1 | ACTCTCTAAGCCACGGAATTAA | CCACAACTCCTCCAATCACAA |
| | | (SEQ ID NO: 19) | (SEQ ID NO: 20) |
| 10561 | IFI44.1 | CCATCGCTGAAGGACAGAA | GCTATCCACATGAGTGAGCAAA |
| | | (SEQ ID NO: 21) | (SEQ ID NO: 22) |
| 3433 | IFIT2.1 | TGGGGGACCAAAGTCTAA | TCTCTGCCCTCGTCTCAA |
| | | (SEQ ID NO: 23) | (SEQ ID NO: 24) |
| 3437 | IFIT4.1,2 | TGGCTACCTCTATCACCAGATTT | CAGCATCAGGGACTTCCTTATT |
| | | (SEQ ID NO: 25) | (SEQ ID NO: 26) |
| 3659 | IRF1.1 | CCAGATATCGAGGAGGTGAAA | GCTGCTGAGTCCATCAGAGAA |
| | | (SEQ ID NO: 27) | (SEQ ID NO: 28) |
| 4599 | MX1.1 | AGCCTGATCTGGTGGACAA | TGTGATGAGGTCGCTGGTAA |
| | | (SEQ ID NO: 29) | (SEQ ID NO: 30) |
| 4600 | MX2.1 | GCACAGTGCCACCACAAA | CAGGGAGTCGATGAGGTCAA |
| | | (SEQ ID NO: 31) | (SEQ ID NO: 32) |
| 4940 | OAS3.1 | CAAGGCCTCAAGAGTCAGTAA | GCGCTCGCATCTCATCAA |
| | | (SEQ ID NO: 33) | (SEQ ID NO: 34) |
| 5610 | PRKR.1 | GGAAAGCGAACAAGGAGTAA | CATCCCGTAGGTCTGTGAAA |
| | | (SEQ ID NO: 35) | (SEQ ID NO: 36) |
| 6772 | STAT1.2 | AAGTCATGGCTGCTGAGAA | GCTGTGATGGCGATAGCAA |
| | | (SEQ ID NO: 37) | (SEQ ID NO: 38) |
| 6772 | STAT1.1,2 | ACGCACACAAAAGTGATGAA | CATGGTGGAGTCAGGAAGAA |
| | | (SEQ ID NO: 39) | (SEQ ID NO: 40) |
| 9830 | TRIM14.1,2,3,4 | GGCTAATGCAGAGTCAAGTAAA | GGCCGTGTATGCCTGAA |
| | | (SEQ ID NO: 41) | (SEQ ID NO: 42) |

Table 5 presents additional updated information about the markers listed in Table 4 including updated Locus Link symbol designations and GenBank Accession Nos. for each of the markers identified. This material including the nucleotide sequences associated with each GenBank Accession No. are hereby incorporated by reference in their entirety for all purposes.

TABLE 5

| LL_Symbol Update | Old LL_Symbol | LOCUS_ID | NM |
|---|---|---|---|
| ADAR | ADAR | 103 | NM_001111 |
| ADAR | ADAR | 103 | NM_015840 |
| ADAR | ADAR | 103 | NM_015841 |
| CXCL10 | CXCL10 | 3627 | NM_001565 |
| G1P2 | G1P2 | 9636 | NM_005101 |
| G1P3 | G1P3 | 2537 | NM_002038 |
| G1P3 | G1P3 | 2537 | NM_022872 |
| G1P3 | G1P3 | 2537 | NM_022873 |
| IFI27 | IFI27 | 3429 | NM_005532 |
| IFI44 | IFI44 | 10561 | NM_006417 |
| IFIT3 | IFIT4 | 3437 | NM_001549 |
| IFIT2 | IFIT2 | 3433 | NM_001547 |
| IRF1 | IRF1 | 3659 | NM_002198 |
| MX1 | MX1 | 4599 | NM_002462 |
| MX2 | MX2 | 4600 | NM_002463 |
| OAS3 | OAS3 | 4940 | NM_006187 |
| PRKR | PRKR | 5610 | NM_002759 |
| STAT1 | STAT1 | 6772 | NM_007315 |
| STAT1 | STAT1 | 6772 | NM_139266 |
| TRIM14 | TRIM14 | 9830 | NM_014788 |
| TRIM14 | TRIM14 | 9830 | NM_033219 |
| TRIM14 | TRIM14 | 9830 | NM_033220 |
| TRIM14 | TRIM14 | 9830 | NM_033221 |

The invention also provides for subsets of at least 2, 3, 4, 5, 6, 7, 8, 9, 10, 11, 12, 13, 14, or 15 genetic markers drawn from the set of markers in Tables 1, 3, and 4 that also can distinguish responder and non-responder phenotypes. Preferably, the number of markers is 10. More preferably, the number of markers is 5.

The invention also provides for subsets of at least 2, 3, 4, 5, 6, 7, 8, 9, 10, 15, 20, 25, 30 or 31 or any number in between of genetic markers in Tables 1 and 3 that also can distinguish responder and non-responder phenotypes. Any of the marker sets provided above may also be used in combination with other markers for IFN response or for any other clinical or physiological condition.

Identification of Markers

The present invention provides sets of markers for the identification of conditions or indications associated with response to IFN treatment in HCV infected patients. In particular, the invention provides for markers that can differentiate between HCV infected patients that will likely respond to IFN treatment versus HCV infected patients that will less likely respond to IFN treatment.

The comparison of markers expression levels in the two patient groups may be accomplished by any means known in the art. For example, expression levels of various markers may be assessed by separation of target polynucleotide molecules (e.g., RNA or cDNA) derived from the markers in agarose or polyacrylamide gels, followed by hybridization with marker-specific oligonucleotide probes. Alternatively, the comparison may be accomplished by the labeling of target polynucleotide molecules followed by separation on a sequencing gel. Polynucleotide samples are placed on the gel such that patient and control or standard polynucleotides are in adjacent lanes. Comparison of expression levels is accomplished visually or by means of densitometer. In a preferred embodiment, the expression of all markers is assessed simultaneously by hybridization to a microarray. In another preferred embodiment, the expression of the markers is assessed by kinetic PCR (RT-PCR). In each approach, markers meeting certain criteria are identified as associated with the IFN response.

A marker is selected in the invention based upon significant difference of expression in a sample as compared to a standard or control condition. Selection may be made based upon either significant up—or down regulation of the marker in the patient sample. Selection may also be made by calculation of the statistical significance (i.e., the p-value) of the correlation between the expression of the marker and the condition or indication. Preferably, both selection criteria are used. Thus, in one embodiment of the present invention, markers associated with IFN response are selected where the markers show both more than two-fold change (increase or decrease) in expression as compared to a standard, and the p-value for the correlation between the existence of viral load and the change in marker expression is no more than 0.01 (i.e., is statistically significant).

The expression of the identified IFN response-related markers is then used to differentiate patients into responder and non-responder phenotypes. In a specific embodiment by way of working examples, using a number of patient samples, markers are identified by calculation of correlation coefficients between the clinical category or clinical parameter(s) and the linear, logarithmic or any transform of the expression ratio across all samples for each individual gene.

Next, the significance of the correlation is calculated. This significance may be calculated by any statistical means by which such significance is calculated. In one method, a set of correlation data is generated using a Monte-Carlo technique to randomize the association between the expression difference of a particular marker and the clinical category. The frequency distribution of markers satisfying the criteria through calculation of correlation coefficients is compared to the number of markers satisfying the criteria in the data generated through the Monte-Carlo technique. The frequency distribution of markers satisfying the criteria in the Monte-Carlo runs is used to determine whether the number of markers selected by correlation with clinical data is significant. Alternatively, the significance of the correlation may be calculated using a semi-supervised principal component approach, a semi-supervised clustering approach, a nearest neighbor classifier approach, or a univariate analysis. See the examples for more details.

Once a marker set is identified, the markers may be rank-ordered in order of significance of discrimination. One means of rank ordering is by the amplitude of correlation between the change in gene expression of the marker and the specific condition being discriminated. Another preferred means is to use a statistical metric.

The rank-ordered marker set may be used to optimize the number of markers in the set used for discrimination. This is accomplished generally in a “leave one out” method as follows. In a first run, a subset, for example 5, of the markers from the top of the ranked list is used to generate a template, where out of X samples, X-1 are used to generate the template, and the status of the remaining sample is predicted. This process is repeated for every sample until every one of the X samples is predicted once. In a second run, additional markers, for example 5, are added, so that a template is now generated from 10 markers, and the outcome of the remaining sample is predicted. This process is repeated until the entire set of markers is used to generate the template. For each of the runs, type 1 error (false negative) and type 2 errors (false positive) are counted; the optimal number of markers is that number where the type 1 error rate, or type 2 error rate, or preferably the total of type 1 and type 2 error rate is lowest.

For prognostic markers, validation of the marker set may be accomplished by additional statistics. A number of statistical models may be used, including Weibull, normal, log-normal, log logistic, log-exponential, or log-Rayleigh (Chapter 12 “Life Testing”, S-PLUS 2000 Guide To Statistics, Vol. 2, p. 368 (2000)).

Sample Collection

In the present invention, target polynucleotide molecules are extracted from a sample taken from a patient who has or has had an HCV infection. The sample may be collected in any clinically acceptable manner, but must be collected such that marker-derived polynucleotides (i.e., RNA) are preserved. mRNA or nucleic acids derived therefrom obtained from the sample may then be analyzed further. For example pairs of oligonucleotides specific for a marker or a set of gene markers (i.e. the markers presented in Tables 1, 3 and 4) may be used to amplify the specific message(s) in the sample. The amount of each message can then be determined or profiled and the correlation with a disease prognosis or probable response to a treatment regime is made.

Alternatively, mRNA or nucleic acids derived therefrom (i.e., cDNA or amplified DNA) are preferably labeled distinguishably from standard or control polynucleotide molecules, and both are simultaneously or independently hybridized to a microarray comprising some or all of the probes to the markers or marker sets or subsets described above. Alternatively, mRNA or nucleic acids derived therefrom may be labeled with the same label as the standard or control polynucleotide molecules, wherein the intensity of hybridization of each at a particular probe is compared. A sample may comprise any clinically relevant tissue sample, such as a formalin fixed paraffin embedded sample, liver biopsy or fine needle aspirate, or a sample of bodily fluid, such as blood, plasma, serum, lymph, ascitic fluid, cystic fluid, or urine.

Methods for preparing total and poly (A)+ RNA are well known and are described generally in Sambrook et at., MOLECULAR CLONING—A LABORATORY MANUAL (2ND ED.), Vols. 1-3, Cold Spring Harbor Laboratory, Cold Spring Harbor, N.Y. (1989)) and Ausubel et al., Current Protocols in Molecular Biology vol. 2, Current Protocols Publishing, New York (1994)). RNA may be isolated by the use of commercially available kits such as the RNeasy mini kit (Qiagen). RNA may be isolated from eukaryotic cells by procedures that involve lysis of the cells and denaturation of the proteins contained therein. RNA may be isolated from formalin-fixed paraffin-embedded using techniques well known in the art. Commercial kits for this purpose may be obtained from Zymo Research, Ambion, Qiagen, or Stratagene.

Additional steps may be employed to remove DNA. Cell lysis may be accomplished with a nonionic detergent, followed by micro-centrifugation to remove the nuclei and hence the bulk of the cellular DNA. In one embodiment, RNA is extracted from cells of the various types of interest using guanidinium thiocyanate lysis followed by CsCl centrifugation to separate the RNA from DNA (Chirgwin et at, Biochemistry 18:5294-5299 (1979)). Poly (A)+ RNA is selected by selection with oligo-dT cellulose (see Sambrook et al, MOLECULAR CLONING—A LABORATORY MANUAL (2ND ED.), Vols. 1-3, Cold Spring Harbor Laboratory, Cold Spring Harbor, N.Y. (1989). Alternatively, separation of RNA from DNA can be accomplished by organic extraction, for example, with hot phenol or phenol/chloroform/isoamyl alcohol.

If desired, RNase inhibitors may be added to the lysis buffer. Likewise, for certain cell types, it may be desirable to add a protein denaturation/digestion step to the protocol.

For many applications, it is desirable to preferentially enrich mRNA with respect to other cellular RNAs, such as transfer RNA (tRNA) and ribosomal RNA (rRNA).

Most mRNAs contain a poly (A) tail at their 3′ end. This allows them to be enriched by affinity chromatography, for example, using oligo (dT) or poly (U) coupled to a solid support, such as cellulose or Sephadex™ (see Ausubel et al., CURRENT PROTOCOLS IN MOLECULAR BIOLOGY, vol. 2, Current Protocols Publishing, New York (1994). Once bound, poly (A)+ mRNA is eluted from the affinity column using 2 mM EDTA/0.1% SDS.

The sample of RNA can comprise a plurality of different mRNA molecules, each different mRNA molecule having a different nucleotide sequence. In a specific embodiment, the mRNA molecules in the RNA sample comprise at least 100 different nucleotide sequences. More preferably, the mRNA molecules of the RNA sample comprise mRNA molecules corresponding to each of the marker genes. In another specific embodiment, the RNA sample is a human RNA sample.

Methods of Using IFN Response Marker Sets

Prognostic Methods

The present invention provides sets of markers useful for distinguishing samples from those patients that respond to IFN treatment (responder phenotype) from samples from patients that do not respond to IFN treatment (non-responder phenotype). IFN treatment includes all forms of IFN, such as PEG-IFN. Thus, the invention further provides a method for using these markers to determine whether an individual infected with HCV will or will not respond to IFN treatment. In one embodiment, the invention provides for method of determining whether an individual infected with HCV will respond to IFN treatment comprising (1) comparing the level of expression of the markers listed in Tables 1, 3, and 4 in a sample taken from the individual to the level of the same markers in a standard or control, where the standard or control levels represent those found in an individual who responds to IFN treatment; and (2) determining whether the level of the marker-related polynucleotides in the sample from the individual is significantly different than that of the control, wherein if no substantial difference is found, the patient has a good prognosis and will respond to treatment, and if a substantial difference is found, the patient has a poor prognosis and may not respond to IFN treatment. Persons of skill in the art will readily see that the markers associated with poor prognosis can also be used as controls. In a more specific embodiment, both controls are run. In case the pool is not pure ‘responder’ or ‘non-responder’, a set of experiments of individuals with known outcome should be hybridized against the pool to define the expression templates for the good prognosis and poor prognosis group. Each individual with unknown outcome is compared against the same pool and the resulting expression profile is compared to the templates to predict its outcome.

The invention provides for a method of determining a course of treatment of an HCV infected patient, comprising determining whether the level of expression of the markers of Tables 1, 3 and 4 or a subset thereof, correlates with the level of these markers in a sample representing a responder phenotype expression pattern or a non-responder pattern; and determining a course of treatment. If a responder pattern is found, the patient may be treated with the standard IFN treatment regime. If a non-responder pattern is found, the patient may be treated with an alternative therapy.

There are two ways of obtaining the information to determine if an infected individual will respond to a course of treatment. The first method is an in vitro method that is described in greater detail in the example section. Briefly, a blood sample is obtained from an HCV infected individual and cells in that sample are treated with IFN and the response to treatment is determined. Isolated cells are incubated with IFN for 2-20 hours prior to analysis of expression profiles. If the cells display a responder phenotype expression pattern, the individual from whom the cells were obtained may be put on IFN treatment. If the cells display a non-responder phenotype expression pattern, a different form of treatment may be selected. The second method would be to obtain a blood sample from an individual prior to beginning IFN treatment and a blood sample shortly after (1-5 days e.g. or more preferably 2-72 hours after in vivo administration of IFN) treatment has commenced. The marker expression patterns can be determined in both samples and if a responder genotype expression pattern is seen in the samples obtained from the treated individual, treatment may be continued. If a non-responder phenotype expression pattern is observed, treatment may be terminated and an alternative treatment may be started.

Classification of a sample as “responder phenotype” or “non-responder phenotype” is accomplished substantially as for the diagnostic markers described above, wherein a template is generated to which the marker expression levels in the sample are compared. Where a set of markers has been identified that corresponds to two or more phenotypes, the marker sets can be used to distinguish these phenotypes. For example, the phenotypes maybe the diagnosis and/or prognosis of clinical states or phenotypes associated with other disease conditions, or other physiological conditions, wherein the expression level data is derived from a set of genes correlated with the particular physiological or disease condition.

Improving the Sensitivity to Expression Level Differences

In using the markers disclosed herein, and, indeed, using any sets of markers to differentiate an individual having one phenotype from another individual having a second phenotype, one can compare the absolute expression of each of the markers in a sample to a control; for example, the control can be the average level of expression of each of the markers, respectively, in a pool of individuals. To increase the sensitivity of the comparison, however, the expression level values are preferably transformed in a number of ways.

For example, the expression level of each of the markers can be normalized by the average expression level of all markers the expression level of which is determined, or by the average expression level of a set of control genes. Thus, in one embodiment, the markers are represented by probes on a microarray, and the expression level of each of the markers is normalized by the mean or median expression level across all of the genes represented on the microarray, including any non-marker genes. In a specific embodiment, the normalization is carried out by dividing the median or mean level of expression of all of the genes on the microarray. In another embodiment, the expression levels of the markers is normalized by the mean or median level of expression of a set of control markers. In a specific embodiment, the control markers comprise a set of housekeeping genes. In another specific embodiment, the normalization is accomplished by dividing by the median or mean expression level of the control genes.

The sensitivity of a marker-based assay will also be increased if the expression levels of individual markers are compared to the expression of the same markers in a pool of samples. Preferably, the comparison is to the mean or median expression level of each the marker genes in the pool of samples. Such a comparison may be accomplished, for example, by dividing by the mean or median expression level of the pool for each of the markers from the expression level each of the markers in the sample. This has the effect of accentuating the relative differences in expression between markers in the sample and markers in the pool as a whole, making comparisons more sensitive and more likely to produce meaningful results than the use of absolute expression levels alone. The expression level data may be transformed in any convenient way; preferably, the expression level data for all is log transformed before means or medians are taken.

In performing comparisons to a pool, two approaches may be used. First, the expression levels of the markers in the sample may be compared to the expression level of those markers in the pool, where nucleic acid derived from the sample and nucleic acid derived from the pool are hybridized or amplified by RT-PCR during the course of a single experiment. Such an approach requires that new pool nucleic acid be generated for each comparison or limited numbers of comparisons, and is therefore limited by the amount of nucleic acid available. Alternatively, and preferably, the expression levels in a pool, whether normalized and/or transformed or not, are stored on a computer, or on computer-readable media, to be used in comparisons to the individual expression level data from the sample (i.e., single-channel data).

Thus, the current invention provides the following method of classifying a first cell or organism as having one of at least two different phenotypes, where the different phenotypes comprise a first phenotype and a second phenotype. The level of expression of each of a plurality of genes in a first sample from the first cell or organism is compared to the level of expression of each of said genes, respectively, in a pooled sample from a plurality of cells or organisms, the plurality of cells or organisms comprising different cells or organisms exhibiting said at least two different phenotypes, respectively, to produce a first compared value. The first compared value is then compared to a second compared value, wherein said second compared value is the product of a method comprising comparing the level of expression of each of said genes in a sample from a cell or organism characterized as having said first phenotype to the level of expression of each of said genes, respectively, in the pooled sample. The first compared value is then compared to a third compared value, wherein said third compared value is the product of a method comprising comparing the level of expression of each of the genes in a sample from a cell or organism characterized as having the second phenotype to the level of expression of each of the genes, respectively, in the pooled sample. Optionally, the first compared value can be compared to additional compared values, respectively, where each additional compared value is the product of a method comprising comparing the level of expression of each of said genes in a sample from a cell or organism characterized as having a phenotype different from said first and second phenotypes but included among the at least two different phenotypes, to the level of expression of each of said genes, respectively, in said pooled sample. Finally, a determination is made as to which of said second, third, and, if present, one or more additional compared values, said first compared value is most similar, wherein the first cell or organism is determined to have the phenotype of the cell or organism used to produce said compared value most similar to said first compared value.

In a specific embodiment of this method, the compared values are each ratios of the levels of expression of each of said genes. In another specific embodiment, each of the levels of expression of each of the genes in the pooled sample are normalized prior to any of the comparing steps. In a more specific embodiment, the normalization of the levels of expression is carried out by dividing by the median or mean level of the expression of each of the genes or dividing by the mean or median level of expression of one or more housekeeping genes in the pooled sample from said cell or organism. In another specific embodiment, the normalized levels of expression are subjected to a log transform, and the comparing steps comprise subtracting the log transform from the log of the levels of expression of each of the genes in the sample. In another specific embodiment, the two or more different phenotypes are different stages of a disease or disorder. In still another specific embodiment, the two or more different phenotypes are different prognoses of a disease or disorder. In yet another specific embodiment, the levels of expression of each of the genes, respectively, in the pooled sample or said levels of expression of each of said genes in a sample from the cell or organism characterized as having the first phenotype, second phenotype, or said phenotype different from said first and second phenotypes, respectively, arc stored on a computer or on a computer-readable medium.

In another specific embodiment, the two phenotypes are good prognosis and poor prognosis.

Of course, single-channel data may also be used without specific comparison to a mathematical sample pool. For example, a sample may be classified as having a first or a second phenotype, wherein the first and second phenotypes are related, by calculating the similarity between the expression of at least 2-4 or more markers in the sample, where the markers are correlated with the first or second phenotype, to the expression of the same markers in a first phenotype template and a second phenotype template, by (a) labeling nucleic acids derived from a sample with a fluorophore to obtain a pool of fluorophore-labeled nucleic acids; (b) contacting said fluorophore-labeled nucleic acid with a microarray under conditions such that hybridization can occur, detecting at each of a plurality of discrete loci on the microarray a fluorescent emission signal from said fluorophore-labeled nucleic acid that is bound to said microarray under said conditions; and (c) determining the similarity of marker gene expression in the individual sample to the first and second templates, wherein if said expression is more similar to the first template, the sample is classified as having the first phenotype, and if said expression is more similar to the second template, the sample is classified as having the second phenotype.

Determination of Marker Gene Expression Levels

The expression levels of the marker genes in a sample may be determined by any means known in the art. The expression level may be determined by isolating and determining the level (i.e., amount) of nucleic acid transcribed from each marker gene. Alternatively, or additionally, the level of specific proteins translated from mRNA transcribed from a marker gene may be determined.

The level of expression of specific marker genes can be accomplished by determining the amount of mRNA, or polynucleotides derived therefrom, present in a sample. Any method for determining RNA levels can be used. For example, RNA is isolated from a sample and separated on an agarose gel. The separated RNA is then transferred to a solid support, such as a filter. Nucleic acid probes representing one or more markers are then hybridized to the filter by northern hybridization, and the amount of marker-derived RNA is determined. Such determination can be visual, or machine-aided, for example, by use of a densitometer. Another method of determining RNA levels is by use of a dot-blot or a slot-blot. In this method, RNA, or nucleic acid derived therefrom, from a sample is labeled. The RNA or nucleic acid derived therefrom is then hybridized to a filter containing oligonucleotides derived from one or more marker genes, wherein the oligonucleotides are placed upon the filter at discrete, easily-identifiable locations. Hybridization, or lack thereof, of the labeled RNA to the filter-bound oligonucleotides is determined visually or by densitometer. Polynucleotides can be labeled using a radiolabel or a fluorescent (i.e., visible) label. These examples are not intended to be limiting; other methods of determining RNA abundance are known in the art.

The level of expression of particular marker genes may also be assessed by determining the level of the specific protein expressed from the marker genes. This can be accomplished, for example, by separation of proteins from a sample on a polyacrylamide gel, followed by identification of specific marker-derived proteins using antibodies in a western blot. Alternatively, proteins can be separated by two-dimensional gel electrophoresis systems. Two-dimensional gel electrophoresis is well known in the art and typically involves isoelectric focusing along a first dimension followed by SDS-PAGE electrophoresis along a second dimension. See, e.g., Hames et al, 1990, GEL ELECTROPHORESIS OF PROTINS: A PRACTICAL APPROACH, IRL Press, New York; Shevehenko et al., Proc. Nat Acad. Sci. USA 93:1440-1445 (1996); Saglioeco et al., Yeast 12:1519-1533 (1996); Lander, Science 274:536-539 (1996). The resulting electropherograms can be analyzed by numerous techniques, including mass spectrometry techniques, western blotting and immunoblot analysis using polyclonal and monoclonal antibodies.

Alternatively, marker-derived protein levels can be determined by constructing an antibody microarray in which binding sites comprise immobilized, preferably monoclonal, antibodies specific to a plurality of protein species encoded by the cell genome. Preferably, antibodies are present for a substantial fraction of the marker-derived proteins of interest. Methods for making monoclonal antibodies are well known (see, e.g., Harlow and Lane, 1988, ANTIBODIES: A LABORATORY MANUAL, Cold Spring Harbor, N.Y., which is incorporated in its entirety for all purposes). In one embodiment, monoclonal antibodies are raised against synthetic peptide fragments designed based on genomic sequence of the cell. With such an antibody array, proteins from the cell are contacted to the array, and their binding is assayed with assays known in the art. Generally, the expression, and the level of expression, of proteins of diagnostic or prognostic interest can be detected through immunohistochemical staining of tissue slices or sections.

Finally, expression of marker genes in a number of tissue specimens may be characterized using a “tissue array” (Kononen et al., Nat. Med 4(7):844-7 (1998)). In a tissue array, multiple tissue samples are assessed on the same microarray. The arrays allow in situ detection of RNA and protein levels; consecutive sections allow the analysis of multiple samples simultaneously.

Microarrays and Kinetic RT-PCR Gene Expression Profiling

In one preferred embodiment, polynucleotide microarrays are used to measure expression so that the expression status of each of the markers above is assessed simultaneously. In a specific embodiment, oligonucleotide or cDNA arrays comprising probes hybridizable to the genes corresponding to each of the marker sets described above (i.e., markers to distinguish patients with good versus patients with poor prognosis). The microarrays may comprise probes hybridizable to the genes corresponding to markers listed in Tables 1, 3 or 4. For example, in a specific embodiment, the microarray is a screening or scanning array as described in Altschuler et al., International Publication WO 02/1 8646, published Mar. 7, 2002 and Scherer et al., International Publication WO 02/16650, published Feb. 28, 2002. The scanning and screening arrays comprise regularly-spaced, positionally-addressable probes derived from genomic nucleic acid sequences, both expressed and unexpressed. Such arrays may comprise probes corresponding to a subset of, or all of, the markers listed in Tables 1, 3 or 4, or a subset thereof as described above, and can be used to monitor marker expression in the same way as a microarray containing only markers listed in Tables 1, 3 or 4.

In yet another specific embodiment, the microarray is a commercially available cDNA microarray that comprises at least 2-4 of the markers listed in Tables 1, 3 or 4. Preferably, a commercially-available cDNA microarray comprises all of the markers listed in Tables 1, 3 or 4. However, such a microarray may comprise 5, 10, 15, 25, 50, 100, 150, 200 or more of the markers in any of Tables 1, 3, or 4, up to the maximum number of markers in a Table, and may comprise all of the markers in any one of Tables 1, 3 or 4 and a subset of another of Tables 1 3 or 4, or subsets of each as described above. In a specific embodiment of the microarrays used in the methods disclosed herein, the markers that are all or a portion of Tables 1, 3 or 4 make up at least 50/, 60%, 70%, 80%, 90%, 95% or 98% of the probes on the microarray.

Construction of Microarrays

Microarrays are prepared by selecting probes that comprise a polynucleotide sequence, and then immobilizing such probes to a solid support or surface. For example, the probes may comprise DNA sequences, RNA sequences, or copolymer sequences of DNA and RNA. The polynucleotide sequences of the probes may also comprise DNA and/or RNA analogues, or combinations thereof. For example, the polynucleotide sequences of the probes may be full or partial fragments of genomic DNA. The polynucleotide sequences of the probes may also be synthesized nucleotide sequences, such as synthetic oligonucleotide sequences. The probe sequences can be synthesized either enzymatically in vivo, enzymatically in vitro (e.g., by PCR), or non-enzymatically in vitro.

Microarrays can be made in a number of ways, of which several are described below. However produced, microarrays share certain characteristics. The arrays are reproducible, allowing multiple copies of a given array to be produced and easily compared with each other. Preferably, microarrays are made from materials that are stable under binding (e.g., nucleic acid hybridization) conditions. The microarrays are preferably small, e.g., between 1 cm² and 25 cm², between 12 cm² and 13 cm², or 3 cm². However, larger arrays are also contemplated and may be preferable, e.g., for use in screening arrays. Preferably, a given binding site or unique set of binding sites in the microarray will specifically bind (e.g., hybridize) to the product of a single gene in a cell (e.g., to a specific mRNA, or to a specific cDNA derived there from). However, in general, other related or similar sequences will cross hybridize to a given binding site.

The microarrays of the present invention include one or more test probes, each of which has a polynucleotide sequence that is complementary to a subsequence of RNA or DNA to be detected. Preferably, the position of each probe on the solid surface is known. Indeed, the microarrays are preferably positionally addressable arrays. Specifically, each probe of the array is preferably located at a known, predetermined position on the solid support such that the identity (i.e., the sequence) of each probe can be determined from its position on the array (i.e., on the support or surface). According to the invention, the microarray is an array (i.e., a matrix) in which each position represents one of the markers described herein.

Preparing Probes for Microarrays and Primers for RT-PCR

As noted above, the “probe” to which a particular polynucleotide molecule specifically hybridizes contains a complementary genomic polynucleotide sequence. The probes of the microarray preferably consist of nucleotide sequences of no more than 1,000 nucleotides. In some embodiments, the probes of the array consist of nucleotide sequences of 10 to 1,000 nucleotides. In a preferred embodiment, the nucleotide sequences of the probes are in the range of 10-200 nucleotides in length and are genomic sequences of a species of organism, such that a plurality of different probes is present, with sequences complementary and thus capable of hybridizing to the genome of such a species of organism, sequentially tiled across all or a portion of such genome. Primer sets used in RT-PCR amplification assays to specifically amplify a particular polynucleotide contain polynucleotides that are identical to or complementary to the first strand synthesized in a reverse transcriptase reaction using a specific mRNA as a template. Primers may be 8-50 or more nucleotides in length, preferably 10-30 nucleotides in length and more preferably 15-25 nucleotides in length. Primers in a set used to amplify a specific nucleotide sequence are usually spaced 10-1000 nucleotides apart on that sequence, preferably the primers are spaced 25-500 nucleotides apart and more preferably, 50-250 nucleotides apart.

The probes or primers may comprise DNA or DNA “mimics” (e.g., derivatives and analogues) corresponding to a portion of an organism's genome. In another embodiment, the microarray probes and PCR primers are complementary RNA or RNA mimics. DNA mimics are polymers composed of subunits capable of specific, Watson-Crick-like hybridization with DNA, or of specific hybridization with RNA. The nucleic acids can be modified at the base moiety, at the sugar moiety, or at the phosphate backbone. Exemplary DNA mimics include, e.g., phosphorothioates. DNA can be obtained, e.g., by polymerase chain reaction (PCR) amplification of genomic DNA or cloned sequences. PCR primers are preferably chosen based on a known sequence of the genome that will result in amplification of specific fragments of genomic DNA. Computer programs that are well known in the art are useful in the design of primers with the required specificity and optimal amplification properties, such as Oligo version 5.0 (National Biosciences). It will be apparent to one skilled in the art that controlled robotic systems are useful for isolating and amplifying nucleic acids.

An alternative, preferred means for generating the polynucleotide microarray probes or PCR primers is by synthesis of synthetic polynucleotides or oligonucleotides, e.g., using N-phosphonate or phosphoramidite chemistries (Froehler et at, Nucleic Acid Res. 14:5399-5407 (1986); McBride et at, Tetrahedron Lett. 24:246-248 (1983)). Synthetic sequences are typically between about 10 and about 500 bases in length, more typically between about 15 and about 100 bases, and most preferably between about 40 and about 70 bases in length for probes and 15-25 bases for primers. In some embodiments, synthetic nucleic acids include non-natural bases, such as, but by no means limited to, inosine. As noted above, nucleic acid analogues may be used as binding sites for hybridization. An example of a suitable nucleic acid analogue is peptide nucleic acid (see, e.g., Eghoim et at, Nature 363:566-568 (1993); U.S. Pat. No. 5,539,083).

Probes and primers are preferably selected using an algorithm that takes into account binding energies, base composition, sequence complexity, cross-hybridization binding energies, and secondary structure (see Friend et at, International Patent Publication WO 01105935, published Jan. 25, 2001; Hughes et al, Nat. Biotech. 19:342-7 (2001)).

As used herein, an “amplified polynucleotide” or “amplicon” of the invention is a marker-containing nucleic acid molecule whose amount has been increased at least two fold by an nucleic acid amplification method performed in vitro as compared to its starting amount in a test sample. In other preferred embodiments, an amplified polynucleotide is the result of at least ten fold, fifty fold, one hundred fold, one thousand fold, or even ten thousand fold increase as compared to its starting amount in a test sample. In a typical PCR amplification, a polynucleotide of interest is often amplified at least fifty thousand fold in amount over the unamplified genomic DNA, but the precise amount of amplification needed for an assay depends on the sensitivity of the subsequent detection method used.

Generally, an amplified polynucleotide is at least twenty nucleotides in length. More typically, an amplified polynucleotide is at least thirty nucleotides in length. In a preferred embodiment of the invention, an amplified polynucleotide is at least fifty nucleotides in length. In a more preferred embodiment of the invention, an amplified polynucleotide is at least one hundred nucleotides in length. While the total length of an amplified polynucleotide of the invention can be the entire marker gene of interest, an amplified product is typically no greater than about five hundred nucleotides in length and is preferably between 100 and 300 nucleotides in length.

Attaching Probes to the Solid Surface

The microarray probes are attached to a solid support or surface, which may be made, e.g., from glass, plastic (e.g., polypropylene, nylon), polyacrylamide, nitrocellulose, gel, or other porous or nonporous material. A preferred method for attaching the nucleic acids to a surface is by printing on glass plates, as is described generally by Schena et al, Science 270:467-470 (1995). This method is especially useful for preparing microarrays of Edna (See also, DeRisi et al, Nature Genetics 14:457-460 (1996); Shalon et at, Genome Res. 6:639-645 (1996); and Schena et at, Proc. Nat. Acad. Sci U.S.A. 93:10539-I 1286 (1995)).

A second preferred method for making microarrays is by making high-density oligonucleotide arrays. Techniques are known for producing arrays containing thousands of oligonucleotides complementary to defined sequences, at defined locations on a surface using photolithographic techniques for synthesis in situ (see, Fodor et at, 1991, Science 25 1: 767-773; Pease et at, 1994, Proc. Nat Acad. Sci U.S.A. 91:5022-5026; Lockhart et at, 1996, Nature Biotechnology 14:1675; U.S. Pat. Nos. 5,578,832; 5,556,752; and 5,510,270) or other methods for rapid synthesis and deposition of defined oligonucleotides (Blanchard et at, Biosensors & Bioelectronics I 1:687-690). When these methods are used, oligonucleotides (e.g., 60-mers) of known sequence are synthesized directly on a surface such as a derivatized glass slide. Usually, the array produced is redundant, with several oligonucleotide molecules per BRA. Other methods for making microarrays, e.g., by masking (Maskos and Southern, 1992, Nuc. Acids. Res. 20:1679-1684), may also be used. In principle, and as noted supra, any type of array, for example, dot blots on a nylon hybridization membrane (see Sambrook et al., MOLECULAR CLONING, A LABORATORY MANUAL (2ND ED.), Vols. 1-3, Cold Spring Harbor Laboratory, Cold Spring Harbor, N.Y. (1989)) could be used. However, as will be recognized by those skilled in the art, very small arrays will frequently be preferred because hybridization volumes will be smaller. In one embodiment, arrays are prepared by synthesizing polynucleotide probes on a support. In such an embodiment, polynucleotide probes are attached to the support covalently at either the 3′ or the 5′ end of the polynucleotide inkjet printing device for oligonucleotide synthesis, e.g., using the methods and systems described by Blanchard in U.S. Pat. No. 6,028,189; Blanchard et al., 1996, Biosensors and Bioelectronics 11:687-690; Blanchard, 1998, in SYNTHETIC DNA Assays IN GENETIC ENGINEERING, Vol. 20, 1K. Setlow, Ed., Plenum Press, New York at pages 111-123. Specifically, the oligonucleotide probes in such microarrays are preferably synthesized in arrays, e.g., on a glass slide, by serially depositing individual nucleotide bases in “microdroplets” of a high surface tension solvent such as propylene carbonate. The microdroplets have small volumes (e.g., 100 pL or less, more preferably 50 pL or less) and are separated from each other on the microarray (e.g., by hydrophobic domains) to form circular surface tension wells, which define the locations of the array elements (i.e., the different probes). Microarrays manufactured by this ink-jet method are typically of high density, preferably having a density of at least about 2,500 different probes per 1 cm². The polynucleotide probes are attached to the support covalently at either the 3′ or the 5′ end of the polynucleotide.

Target Polynucleotide Molecules

The polynucleotide molecules which may be analyzed by the present invention (the “target polynucleotide molecules”) may be from any clinically relevant source, but are expressed RNA or a nucleic acid derived there from (e.g., cDNA or amplified RNA derived from cDNA that incorporates an RNA polymerase promoter), including naturally occurring nucleic acid molecules, as well as synthetic nucleic acid molecules. In one embodiment, the target polynucleotide molecules comprise RNA, including, but by no means limited to, total cellular RNA, poly (A) messenger RNA (mRNA) or fraction thereof, cytoplasmic mRNA, or RNA transcribed from cDNA (i.e., cRNA; see, e.g., Linsley & Schelter, U.S. patent application Ser. No. 09/411,074, filed Oct. 4, 1999, or U.S. Pat. Nos. 5,545,522, 5,891,636, or 5,716,785). Methods for preparing total and poly (A) RNA are well known in the art, and are described generally, e.g., in Sambrook et al., MOLECULAR CLONING—A LABORATORY MANUAL (2ND ED.), Vols. 1-3, Cold Spring Harbor Laboratory, Cold Spring Harbor, N.Y. (1989). In one embodiment, RNA is extracted from cells of the various types of interest in this invention using guanidinium thiocyanate lysis followed by CsCl centrifugation (Chirgwin et at, 1979, Biochemistry 18:5294-5299). In another embodiment, total RNA is extracted using a silica gel-based column, commercially available examples of which include RNeasy (Qiagen, Valencia, Calif.) and StrataPrep (Stratagene, La Jolla, Calif.). In an alternative embodiment, RNA is extracted from cells using phenol and chloroform, as described in Ausubel et al., eds., 1989, CURRENT PROTOCOLS IN MOLECULAR BIOLOGY, John Wiley & Sons, Inc., New York, at pp. 13.12.1-13.12.5). RNA may also be extracted from cells or tissues using one of the many commercially available kits. Poly (A) RNA can be selected, e.g., by selection with oligo-dT cellulose or, alternatively, by oligo-dT primed reverse transcription of total cellular RNA. In one embodiment, RNA can be fragmented by methods known in the art, e.g., by incubation with ZnCl₂, to generate fragments of RNA. In another embodiment, the polynucleotide molecules analyzed by the invention comprise cDNA, or PCR products of amplified RNA or cDNA.

In one embodiment, total RNA, mRNA, or nucleic acids derived there from, is isolated from a sample taken from a person infected with HCV. Target polynucleotide molecules that are poorly expressed in particular cells may be enriched using normalization techniques Qonaldo et al., 1996, Genome Res. 6:791-806). As described above, the target polynucleotides are detectably labeled at one or more nucleotides. Any method known in the art may be used to detectably label the target polynucleotides. Preferably, this labeling incorporates the label uniformly along the length of the RNA, and more preferably, the labeling is carried out at a high degree of efficiency. One embodiment for this labeling uses oligo-dT primed reverse transcription to incorporate the label; however, conventional methods of this method are biased toward generating 3′ end fragments. Thus, in a preferred embodiment, random primers (e.g., 9-mers) are used in reverse transcription to uniformly incorporate labeled nucleotides over the full length of the target polynucleotides. Alternatively, random primers may be used in conjunction with PCR methods or T7 promoter-based in vitro transcription methods in order to amplify the target polynucleotides. In a preferred embodiment, the detectable label is a luminescent label. For example, fluorescent labels, bio-luminescent labels, chemi-luminescent labels, and calorimetric labels may be used in the present invention. In a highly preferred embodiment, the label is a fluorescent label, such as a fluorescein, a phosphor, a rhodamine, or a polymethine dye derivative. Examples of commercially available fluorescent labels include, for example, fluorescent phosphoramidites such as FluorePrime (Amersham Pharmacia, Piscataway, N.J.), Fluoredite (Millipore, Bedford, Mass.), FAM (ABI, Foster City, Calif.), and Cy3 or Cy5 (Amersham Pharmacia, Piscataway, N.J.). In another embodiment, the detectable label is a radiolabeled nucleotide.

In a further preferred embodiment, target polynucleotide molecules from a patient sample are labeled differentially from target polynucleotide molecules of a standard. The standard can comprise target polynucleotide molecules from normal individuals (i.e., those not infected with HCV). In a highly preferred embodiment, the standard comprises target polynucleotide molecules pooled from samples from normal individuals. In another embodiment, the target polynucleotide molecules are derived from the same individual, but are taken at different time points, and thus indicate the efficacy of a treatment by a change in expression of the markers, or lack thereof, during and after the course of treatment (i.e., IFN treatment). In this embodiment, different time points are differentially labeled.

Hybridizatiqn to Microarrays

Nucleic acid hybridization and wash conditions are chosen so that the target polynucleotide molecules specifically bind or specifically hybridize to the complementary polynucleotide sequences of the array, preferably to a specific array site, wherein its complementary DNA is located. Arrays containing double-stranded probe DNA situated thereon are preferably subjected to denaturing conditions to render the DNA single-stranded prior to contacting with the target polynucleotide molecules. Arrays containing single-stranded probe DNA (e.g., synthetic oligodeoxyribonucleic acids) may need to be denatured prior to contacting with the target polynucleotide molecules, e.g., to remove hairpins or dimers which form due to self complementary sequences. Optimal hybridization conditions will depend on the length (e.g., oligomer versus polynucleotide greater than 200 bases) and type (e.g., RNA, or DNA) of probe and target nucleic acids. One of skill in the art will appreciate that as the oligonucleotides become shorter, it may become necessary to adjust their length to achieve a relatively uniform melting temperature for satisfactory hybridization results. General parameters for specific (i.e., stringent) hybridization conditions for nucleic acids are described in Sambrook et al., MOLECULAR CLONING—A LABORATORY MANUAL. (2nd ED.), Vols. 1-3, Cold Spring Harbor Laboratory, Cold Spring Harbor, N.Y. (1989), and in Ausubel et al., CURRENT PROTOCOLS IN MOLECULAR BIOLOGY, vol. 2, Current Protocols Publishing, New York (1994). Typical hybridization conditions for the cDNA microarrays of Schena et al. are hybridization in 5×SSC plus 0.2% SDS at 65° C. for four hours, followed by washes at 25° C. in low stringency wash buffer (1×SSC plus 0.2% SDS), followed by 10 minutes at 25° C. in higher stringency wash buffer (0.1×SSC plus 0.2% SDS) (Schena et at, Proc. Natl. Acad. Sci. U.S.A., 93:106 14 (1993)). Useful hybridization conditions are also provided in, e.g., Tijessen, 1993, HYBRIDIZATION WITH NUCLEIC ACID PROBES, Elsevier Science Publishers B.V.; and Krieka, 1992, NONISOTOPIC DNA PROBE TECHNIQUES, Academic Press, San Diego, Calif.

Particularly preferred hybridization conditions include hybridization at a temperature at or near the mean melting temperature of the probes (e.g., within S° C., more preferably within 2° C.) in 1 M NaCl, 50 mM MES buffer (pH 6.5), 0.5% sodium sarcosine and 30% formamide.

Kinetic RT-PCR

Kinetic RT-PCR may be performed using a variety of probes, buffers and PCR machines. Approaches to RT-PCR are described by Mackay et al., Nucleic Acids Research Vol. 30:1292-1305 (2002) and Kang et al., Nucleic Acids Research Vol. 28n No 2,:1-8 (2000) each of which is incorporated by reference in their entirety.

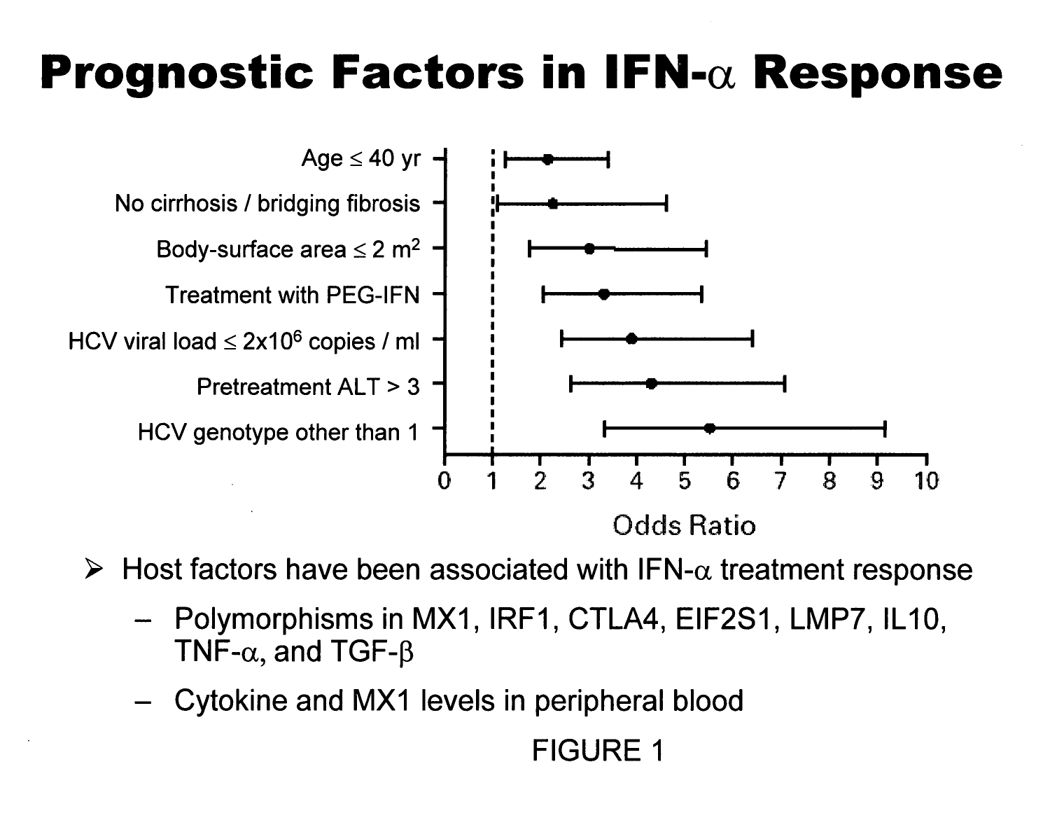

The polymerase chain reaction (PCR) (Freymuth, F. et al., (1995) J. Clin. Microbiol., 33:3352-3355, Mullis, K. B. et al., (1987) Methods Enzymol., 155:335-350) has been used as the new gold standard for detecting a wide variety of templates across a range of scientific specialties, including virology. The method utilizes a pair of synthetic oligonucleotides or primers, each hybridizing to one strand of a double-stranded DNA (dsDNA) target, with the pair spanning a region that will be exponentially reproduced. The hybridized primer acts as a substrate for a DNA polymerase (most commonly derived from the hemophilic bacterium Thermus aquaticus and called Taq), which creates a complementary strand via sequential addition of deoxynucleotides. The process can be summarized in three steps: (i) dsDNA separation at temperatures >90° C., (ii) primer annealing at 50-75° C., and (iii) optimal extension at 72-78° C. (FIG. 1A). The rate of temperature change or ramp rate, the length of the incubation at each temperature and the number of times each set of temperatures (or cycle) is repeated are controlled by a programmable thermal cycler. Current technologies have significantly shortened the ramp times using electronically controlled heating blocks or fan-forced heated air flows to moderate the reaction temperature. Consequently, PCR is displacing some of the gold standard cell culture and serological assays (Niubo, J. et al., (1994). J. Clin. Microbiol., 32:1119-1120). Existing combinations of PCR and detection assays (called ‘conventional PCR’ here) have been used to obtain quantitative data with promising results. However, these approaches have suffered from the laborious post-PCR handling steps required to evaluate the amplicon (Ouatelli, J. C. et al., (1989) Clin. Microbiol. Rev., 2: 217-226).

Traditional detection of amplified DNA relies upon electrophoresis of the nucleic acids in the presence of ethidium bromide and visual or densitometric analysis of the resulting bands after irradiation by ultraviolet light (Kidd, I. M. et al., (2000) J. Virol. Methods, 87:177-1811). Southern blot detection of amplicon using hybridization with a labeled oligonucleotide probe is also time consuming and requires multiple PCR product handling steps, further risking a spread of amplicon throughout the laboratory (Holland, P. M. et al., (1991) Proc. Natl. Acad. Sci. USA, 88:7276-7280). Alternatively, PCR—ELISA may be used to capture amplicon onto a solid phase using biotin or digoxigenin-labeled primers, oligonucleotide probes (oligoprobes) or directly after incorporation of the digoxigenin into the amplicon (van der Vliet, G. M. E., et al., J. Clin. Microbiol., 31:665-670, Keller, O. H. et al., (1990) J. Clin. Microbiol., 28:1411-1416, Kemp, D. J. et al., (1990) Gene, 94:223-228, Kox, L. F. F. et al., (1996) J. Clin. Microbiol., 34:2117-2120, Dekoneoko, A. et al., (1997) Clin. Diag. Virol., 8:113-121, Watzinger, F. et al., (2001) Nucleic Acids Res., 29:e52.). Once captured, the amplicon can be detected using an enzyme-labeled avidin or anti-digoxigenin reporter molecule similar to a standard ELISA format.

The possibility that, in contrast to conventional assays, the detection of amplicon could be visualized as the amplification progressed was a welcome one (Lomeli, H. et al., (1989) Clin. Chem., 35:1826-1831). This approach has provided a great deal of insight into the kinetics of the reaction and it is the foundation of kinetic or ‘real-time’ PCR (FIG. 1B) (Holland, P. M. et al., (1991) Proc. Natl. Acad. Sci. USA, 88:7276-7280, Lee, L. O. et al., (1993) Nucleic Acids Res., 21:3761-3766, Livak, K. J. et al., (1995) PCR Methods Appl., 4:357-362, Heid, C. A. et al., (1996) Genome Res., 6:986-994, Gibson, U. E. M. et al., (1996) Genome Res., 6:995-1001). Real-time PCR has already proven itself valuable in laboratories around the globe, building on the enormous amount of data generated by conventional PCR assays.

The monitoring of accumulating amplicon in real time has been made possible by the labeling of primers, probes or amplicon with fluorogenic molecules. This chemistry has clear benefits over radiogenic oligoprobes that include an avoidance of radioactive emissions, ease of disposal and an extended shelf life (Matthews, J. A. et al., (1988) Anal. Biochem., 169:1-25).

The increased speed of real-time PCR is largely due to reduced cycle times, removal of post-PCR detection procedures and the use of fluorogenic labels and sensitive methods of detecting their emissions (Wittwer, C. T. et al., (1990) Anal. Biochem., 186:328-331, Wittwer, C. T. et al., (1997) Biotechniques, 22:176-181). The reduction in amplicon size generally recommended by the creators of commercial real-time assays may also play a role in this speed, however we have shown that decreased product size does not necessarily improve PCR efficiency (Nitsche, A et al., (2000) J. Clin. Microbiol., 38:2734-2737).

The disadvantages of using real-time PCR in comparison with conventional PCR include the inability to monitor amplicon size without opening the system, the incompatibility of some platforms with some fluorogenic chemistries, and the relatively restricted multiplex capabilities of current applications. Also, the start-up expense of real-time PCR may be prohibitive when used in low-throughput laboratories. These shortcomings are mostly due to limitations in the system hardware or the available fluorogenic dyes or ‘fluorophores’, both of which will be discussed in more detail.

Because most of the popular real-time PCR chemistries depend upon the hybridization of an oligoprobe to its complementary sequence on one of the strands of the amplicon, the use of more of the primer that creates this strand is beneficial to the generation of an increased fluorescent signal (Gyllensten, U. B. et al., (1988) Proc. Natl. Acad. Sci. USA, 85:7652-7656). Asymmetric PCR, as this is known, has been shown to produce improved fluorescence from a hairpin oligoprobe PCR (Poddar, S. K. (2000) Mol. Cell. Probes, 14:25-32) and we have found it directly applicable to other oligoprobe-hybridization assays.

The most commonly used fluorogenic oligoprobes rely upon fluorescence resonance energy transfer (FRET) between fluorogenic labels or between one fluorophore and a dark or ‘black-hole’ non-fluorescent quencher (NFQ), which disperses energy as heat rather than fluorescence. FRET is a spectroscopic process by which energy is passed between molecules separated by 10-100 A that have overlapping emission and absorption spectra (Stryer, L. et al., (1967) Proc. Natl. Acad. Sci. USA, 58:719-726, Clegg, R. M. (1992) Methods Enzymol., 211:353-388). Forster primarily developed the theory behind this process: the mechanism is a non-radiative induced-dipole interaction (Forster, T. (1948) Ann. Phys., 6:55-75). The efficiency of energy transfer is proportional to the inverse sixth power of the distance (R) between the donor and acceptor (11R⁶) fluorophores (Selvin, P. (1995) Methods Enzymol., 246:300-334, Didenko, V. V. (2001) Biotechniques, 31:1106-1121).

Post-amplification manipulation of the amplicon is not required for real-time PCR, therefore these assays are described as ‘closed’ or homogeneous systems. The advantages of homogeneous systems include a reduced result turnaround, minimization of the potential for carry-over contamination and the ability to closely scrutinize the assay's performance (Higuchi, R. et al., (1993) Biotechnology (NY), 11: 1026-1030).

Amplicon Detection

There are several major chemistries currently in use in RT-PCR, and they can be classified into amplicon sequence specific or non-specific methods of real-time PCR detection (Whitcombe, D. et al., (1999) Nat. Biotechnol., 17:804-807). Each of the chemistries has an associated nomenclature to describe the fluorescent labels; however, for general discussion, fluorophore will continue to be used to describe these moieties. Although this review focuses on the use of these chemistries in real-time applications, they can also be used as a label for end-point amplicon detection.

DNA-Binding Fluorophores

The basis of the sequence non-specific detection methods is the DNA-binding fluorogenic molecule. Included in this group are the earliest and simplest approaches to real-time PCR. Ethidium bromide (Higuchi, R. et al., (1992) Biotechnology (NY), 10:413-417), YO-PRO-1 (Ishiguro, T. et al., (1995) Anal. Biochem., 229:207-213, Tseng, S. Y. et al., (1997) Anal. Biochem., 245:207-212) and SYBR® green 1 (Morrison, T. M. et al., (1998) Biotechniques, 24:954-962) all fluoresce when associated with dsDNA which is exposed to a suitable wavelength of light. This approach requires less specialist knowledge than the design of fluorogenic oligoprobes, is less expensive and does not suffer when the template sequence varies, which may abrogate hybridization of an oligoprobe (Komurian-Pradel, F. et al., (2001) J. Virol. Methods, 95:111-119). Formation of primer-dimer (Chou, Q. et al., (1992) Nucleic Acids Res., 20:1717-1723) is common and, together with the formation of specific products, is strongly associated with entry of the PCR into the plateau phase (FIG. IB) (Halford, W. P. (1999) Nat. Biotechnol, 17:835, Halford, W. P. et al., (1999) Anal. Biochem., 266:181-191). Association of a DNA-binding fluorophore with primer-dimer or other non-specific amplification products can confuse interpretation of the results. Adding a short, higher temperature incubation after the extension step in which fluorescence data are acquired minimizes the contribution of these products to the fluorescence signal (Pfaffl, M. (2001) In Meuer, S. et al., (ed), Rapid Cycle Real-Time PCR: Methods and Applications. Springer, Berlin, pp. 281-291). The problem of primer-dimer can also be addressed using software capable of fluorescent melting curve analysis. This method makes use of the temperature at which the dsDNA amplicon is denatured (T₀). The shorter primer-dimer can be discriminated by its reduced T₀ compared with the full-length amplicon. Analysis of the melting curves of amplicon in the presence of SYBR® green 1 has demonstrated that the practical sensitivity of DNA-binding fluorophores is limited by non-specific amplification at low initial template concentrations.

DNA binding fluorophores also increase the Tm and broaden the melting transition, requiring substantial sequence change to produce a shift in the Tm. Oligoprobes are able to discriminate single point mutations using the temperature at which 50% of oligoprobe-target duplexes separate (Wetmur, J. G. (1991). Crit. Rev. Biochem. Mol. Biol., 26:227-259). This temperature is called the melting temperature (TM) and it is dependent upon the concentration of the dsDNA, its length, nucleotide sequence and the solvent composition, and is often confused with Tm (Ririe, K. M. et al., (1997) Anal. Biochem., 245:154-160).

Linear Oligoprobes

The use of a pair of adjacent, fluorogenic hybridization oligoprobes was first described in the late 1980s (Heller, M. J. et al. (1985) In Kingsbury, D. T. and Falkow, S. (eds.), Rapid Detection and Identification of infectious Agents. Academic Press, New York, pp. 245-256, Cardullo, R. A. et al., (1988) Proc. Natl. Acad. Sci. USA, 85:8790-8794) and, now known as ‘HybProbes’, they have become the method of choice for the LightCycler™ (Roche Molecular Biochemicals, Germany), a capillary-based, microvolume fluorimeter and thermocycler with rapid temperature control (Wittwer, C. T. et al., (1997) Biotechniques, 22:176-181, Wittwer, C. T. et al., (1997) Biotechniques, 22:130-138). The upstream oligoprobe is labeled with a 3′ donor fluorophore (FITC) and the downstream probe is commonly labeled with either a LightCycler Red 640 or Red 705 acceptor fluorophore at the 5′ terminus so that when both oligoprobes are hybridized, the two fluorophores are located within 10 nt of each other, sometimes attracting the name ‘kissing’ probes. The plastic and glass composite capillaries are optically clear and act as cuvettes for fluorescence analysis, as well as facilitating rapid heat transfer. Capillaries are rotated past a blue light-emitting diode and fluorescence is monitored by three photodetection diodes with different wavelength filters. The temperature is varied by rapidly heating and cooling air using a heating element and fan which produce ramp rates of 20° C./s, prolonging polymerase survival (Weis, J. H. et al., (1992) Trends Genet., 8:263-264). Additionally, because the oligoprobes are not significantly hydrolyzed during amplification (Bustin, S. A. (2000) J. Mol. Endocrinol., 25:169-193) and the LightCycler is able to monitor the changes in fluorescence emission during denaturation of the adjacent oligoprobes from their amplicon, this system can perform single tube genotyping. This capability, which makes use of fluorescent melting curve analysis, provides significant information about the sequence to which the oligoprobes are binding. Mutation(s) under one or both oligoprobes can be determined by the decrease in melting temperature that they incur due to destabilization of the oligoprobe/target duplex. This has imparted significant improvements in speed upon the diagnosis of genetic disease as well as a growing number of multiplex PCR approaches for the detection of related viral pathogens. Despite the fact that the hybridization does not reach equilibrium using these ramp rates, the apparent TM values are both reproducible and characteristic of a given probe/target duplex (Gundry, C. N. et al., (1999) Genet. Test, 3:365-370).

When comparing signals from the different chemistries, the destruction of nuclease oligoprobes continues despite a plateau in product accumulation whereas SYBR® green I fluorescence in the no template control generally increases non-specifically during later cycles. Adjacent oligoprobe fluorescence begins to decrease as the rate of collision between the growing numbers of complementary amplicon strands increases favoring the formation of dsDNA over the hybridization of oligoprobe to its target DNA strand. Additionally, there is the possibility that some oligoprobe is consumed by sequence-related endonuclease activity (Wilhelm, J. et al., (2001) Biotechniques, 30:1052-1062, Lyamichev, V. et al., (1993) Science, 260:778-783). All three oligoprobe chemistries (SYBR® Green I, nuclease and adjacent oligoprobes) seem capable of detecting amplified product with approximately the same sensitivity (Wittwer, C. T. et al., (1997) Biotechniques, 22:176-181).

Combinations of the above approaches are new appearing as more users of the instrumentation become familiar with the concepts behind real-time PCR and contribute to the literature. If a sequence-specific, fluorophore-labeled linear oligoprobe is added to a SYBR® green 1 mix, currently called the Bi-probe system, FRET will occur and an additional layer of specificity can be obtained (Cardullo, R. A. et al., (1988) Proc. Natl. Acad. Sci. USA, 85:8790-8794, Brechtbuehl, K. et al., (2001) J. Virol. Methods, 93:105-113, Walker, R. A. et al., (2001) J. Clin. Microbiol., 39:1443-1448). An assay using a BODIPY® FL-labeled oligoprobe was adapted to run in the LightCycler using a f-globin target sequence (Kurata, S. et al., (2001) Nucleic Acids Res., 29:e34). The probe was designed so that the fluorophore was located on a terminal cytosine and was quenched by proximity with a complementary guanine. The assay demonstrated that quenching varies linearly with the concentration of template across a defined concentration range. The commonly used fluorophore FITC is inherently quenched by deoxyguanosine nucleotides. The level of quenching can be increased if more guanines are present or a single guanine is located in the first overhang position, 1 nt beyond the fluorophore-labeled terminus of the probe. This approach to amplicon detection is easier to design than fluorogenic oligoprobes, simpler to synthesize and use in real-time PCR and does not require a DNA polymerase with nuclease activity (Crockett, A. O. et al., (2001) Anal. Biochem., 290:89-97).

The light-up probe is a peptide nucleic acid to which the asymmetric cyanine fluorophore thiazole orange is attached (Svanvik, N. et al., (2001) Anal. Biochem., 281:26-35). When hybridized with a nucleic acid target, either as a duplex or triplex, depending on the oligoprobe's sequence, the fluorophore becomes strongly fluorescent. These probes do not interfere with the PCR, do not require conformational change, are sensitive to single nucleotide mismatches allowing fluorescence melting analysis, and because a single reporter is used, a direct measurement of fluorescence can be made instead of the measurement of a change in fluorescence between two fluorophores (Svanvik, N. et al., (2001) Anal. Biochem., 281:26-35, Isacsson, J. et al., (2000) Mol. Cell Probes, 14:321-328). However, non-specific fluorescence has been reported during later cycles using these probes (Svanvik, N. et al., (2000) Anal. Biochem., 287:179-1 82).

5′ Nuclease Oligoprobes

In the late 1980s homogeneous assays were few and far between, but rapid advances in thermocycler instrumentation and the chemistry of nucleic acid manipulation have since made these assays commonplace. The success of these assays revolves around a signal changing in some rapid and measurable way upon hybridization of a probe to its target (Morrison, L. E. et al., (1989) Anal, Biochem., 183:231-244). By using an excess, the time required for hybridization of an oligoprobe to its target, especially when the amount of that target has been increased by PCR or some other amplifying process, is significantly reduced (Wetmur, J. G. (1991). Crit. Rev. Biochem. Mol. Biol., 26:227-259, Morrison, L. E. et al., (1989) Anal, Biochem., 183:231-244). In 1991, Holland cit al. (Holland, P. M. et al., (1991) Proc. Natl. Acad. Sci. USA, 88:7276-7280) described a technique that was to form the foundation for homogeneous PCR using fluorogenic oligoprobes. Amplicon was detected by monitoring the effect of Taq DNA polymerase's 5′-3′ endonuclease activity on specific oligoprobe/target DNA duplexes. The radiolabeled products were examined using thin layer chromatography and the presence or absence of hydrolysis was used as an indicator of duplex formation. These oligoprobes contained a 3′ phosphate moiety, which blocked their extension by the polymerase, but otherwise had no affect on the amplicon's yield.

The desirable criteria for an oligoprobe label are (i) easy attachment of the label to DNA, (ii) detectability at low concentrations, (iii) detectability using simple instrumentation, (iv) production of an altered signal upon specific hybridization, (v) biological safety, (vi) stability at elevated temperatures and (vii) an absence of interference with the activity of the polymerase (Holland, P. M. et al., (1991) Proc. Natl. Acad. Sci. USA, 88:7276-7280, Matthews, J. A. et al., (1988) Anal. Biochem., 169:1-25).

An innovative approach used nick-translation PCR in combination with dual-fluorophore labeled oligoprobes (Lee, L. O. et al., (1993) Nucleic Acids Res., 21:3761-3766). In the first truly homogenous assay of its kind, one fluorophore was added to the 5′ terminus and one to the middle of a sequence specific oligonucleotide probe. When in such close proximity, the 5′ reporter fluorophore (6-carboxy-fluoroscein) transferred laser-induced excitation energy by FRET to the 3′ quencher fluorophore (6-carboxy-tetramethyl-rhodamine; TAMRA), which reduced the lifetime of the reporter's excited state by taking its excess energy and emitting it as a fluorescent signal of its own. TAMRA emitted the new energy at a wavelength that was monitored but not utilized in the presentation of data. However, when the oligoprobe hybridized to its template, the fluorophores were released due to hydrolysis of the oligoprobe component of the probe/target duplex. Once the labels were separated, the reporter's emissions were no longer quenched and the instrument monitored the resulting fluorescence. These oligoprobes have been called 5′ nuclease, hydrolysis or TaqMan® oligoprobes. Nuclease oligoprobes have design requirements that are applicable to the other linear oligoprobe chemistries, including (i) a length of 20-40 nt, (ii) a GC content of 40-60%, (iii) no runs of a single nucleotide, particularly 0, (iv) no repeated sequence motifs, (v) an absence of hybridization or overlap with the forward or reverse primers and (vi) a TM at least 5° C. higher than that of the primers, to ensure the oligoprobe has bound to the template before extension of the primers can occur (Landt, O. (2001) In Meuer, S. et al., (eds.), Rapid Cycle Real-time PCR: Methods and Applications. Springer Verlag, Germany, pp. 35-41).

This technology, however, required the development of a platform to excite and detect fluorescence as well as perform thermal cycling. A charge-coupled device had been described in 1992 for the quantification of conventional reverse transcription (RT)-PCR products (Nakayama, H. et al., (1992) Nucleic Acids Res., 20:4939). In 1993 this approach was combined with a thermal cycler resulting in the first real-time PCR fluorescence excitation and detection platform (Higuchi, R. et al., (1993) Biotechnology (NY), 11: 1026-1030). To date, the ABI Prism® 7700 sequence detection system (Perkin Elmer Corporation/Applied Biosystems, USA) has been the main instrument used for 5′ nuclease oligoprobes. Non-PCR related fluorescence fluctuations have been normalized using a non-participating or ‘passive’ internal reference fluorophore (6-carboxy-N,N, N′,N′-tetramethylrhodamine; ROX). The corrected values, obtained from a ratio of the emission intensity of the reporter signal and ROX, are called RQ˜. To further control amplification fluctuations, the fluorescence from a ‘no-template’ control reaction (RQj is subtracted from RQ˜ resulting in the ARQ value that indicates the magnitude of the signal generated for the given PCR (Gelmini, S. et al., (1997) Clin. Chem., 43:752-758).

The fractional cycle number at which the real-time fluorescence signal mirrors progression of the reaction above the background noise was used as an indicator of successful target amplification (Wilhelm, J. et al., (2001) Clin. Chem., 46:1738-1743). This threshold cycle (CT) is defined as the PCR cycle in which the gain in fluorescence generated by the accumulating amplicon exceeds 10 standard deviations of the mean baseline fluorescence, using data taken from cycles 3 to 15 (Jung, R. et al., (2000) Clin. Chem. Lab. Med., 38:833-836). The CT is proportional to the number of target copies present in the sample (Gibson, U. E. M. et al., (1996) Genome Res., 6:995-1001).

A recent improvement to the nuclease oligoprobe has resulted in the minor groove binding (MGB) oligoprobes. This chemistry replaces the standard TAMRA quencher with an NFQ and incorporates a molecule that stabilizes the oligoprobe-target duplex by folding into the minor groove of the dsDNA (Kutyavin, I. V. et al., (2000) Nucleic Acids Res., 28:655-661). This allows the use of very short (14 nt) oligoprobes, which are ideal for detecting single nucleotide polymorphisms (SNPs). A related use of dual labeled oligonucleotide sequences has been to provide the signal-generating portion of the DNA-PCR system (Todd, A. V. et al., (2000) Clin. Chem., 46:625-630). Here, the reporter and quencher are separated after cleavage of the probe by a DNAzyme, which is created during PCR as the complement of an antisense DNAzyme sequence included in the 5′ tail of one of the primers. Upon cleavage, the dual labeled substrate releases the fluorophores and generates a signal in an analogous manner to the 5′ nuclease probe.

Hairpin Oligoprobes

Molecular beacons were the first hairpin oligoprobes and are a variation of the dual-labeled nuclease oligoprobe. The hairpin oligoprobe's fluorogenic labels are called fluorophore and quencher, and they are positioned at the termini of the oligoprobe. The labels are held in close proximity by distal stem regions of homologous base pairing deliberately designed to create a hairpin structure which results in quenching either by FRET or a direct energy transfer by a collisional mechanism due to the intimate proximity of the labels (Tyagi, S. et al., (1998) Nat. Biotechnol., 16:49-53). In the presence of a complementary sequence, designed to occur within the bounds of the primer binding sites, the oligoprobe will hybridize, shifting into an open configuration. The fluorophore is now spatially removed from the quencher's influence and fluorescent emissions are monitored during each cycle (Tyagi, S. et al., (1996) Nat. Biotechnol, 14:303-308). The occurrence of a mismatch between a hairpin oligoprobe and its target has a greater destabilizing effect on the duplex than the introduction of an equivalent mismatch between the target and a linear oligoprobe. This is because the hairpin structure provides a highly stable alternate conformation. Therefore, hairpin oligoprobes have been shown to be more specific than the more common linear oligoprobes making them ideal candidates for detecting SNPs (Tyagi, S. et al., (1998) Nat. Biotechnol., 16:49-53). The quencher, 4-(4′-dimethylamino-phenylazo)-benzene (DABCYL), differs from that described for the nuclease oligoprobes because it is an NFQ.

The wavelength-shifting hairpin probe is a recent improvement to this chemistry which makes use of a second, harvesting fluorophore. The harvester passes excitation energy acquired from a blue light source and releases it as fluorescent energy in the far-red wavelengths. The energy can then be used by a receptive ‘emitter’ fluorophore that produces light at characteristic wavelengths. This offers the potential for improved multiplex real-time PCR and SNP analysis, using currently available instruments (Tyagi, S. et al., (2000) Nat. Biotechnol., 18:1191-1196). Because the function of these oligoprobes depends upon correct hybridization of the stem, accurate design is crucial to their function (Bustin, S. A. (2000) J. Mol. Endocrinol., 25:169-193).

Self-Fluorescing Amplicon

The self-priming amplicon is similar in concept to the hairpin oligoprobe, except that the label becomes irreversibly incorporated into the PCR product. Two approaches have been described: sunrise primers (now commercially called Amplifluor™ hairpin primers) and scorpion primers (Whitcombe, D. et al., (1999) Nat. Biotechnol., 17:804-807, Whitcombe, D. et al., (1999) Nat. Biotechnol., 17:804-807). The sunrise primer consists of a 5′ fluorophore and a DABCYL NFQ. The labels are separated by complementary stretches of sequence that create a stem when the sunrise primer is closed. At the 3′ terminus is a target-specific primer sequence. The sunrise primer's sequence is intended to be duplicated by the nascent complementary strand and, in this way, the stem is destabilized, the two fluorophores are held −20 nt (70 A) apart and the fluorophore is free to emit its excitation energy for monitoring (Whitcombe, D. et al., (1999) Nat. Biotechnol., 17:804-807). This system could suffer from non-specific fluorescence due to duplication of the sunrise primer sequence during the formation of primer-dimer.

The scorpion primer is almost identical in design except for an adjacent hexethylene glycol molecule that blocks duplication of the signaling portion of the scorpion. In addition to the difference in structure, the function of scorpion primers differs slightly in that the 5′ region of the oligonucleotide is designed to hybridize to a complementary region within the amplicon. This hybridization forces the labels apart disrupting the hairpin and permitting emission in the same way as hairpin probes (Whitcombe, D. et al., (1999) Nat. Biotechnol., 17:804-807).

Quantitation

The majority of diagnostic PCR assays reported to date have been used in a qualitative, or ‘yes/no’ format. The development of real-time PCR has brought true quantitation of target nucleic acids out of the pure research laboratory and into the diagnostic laboratory.

Determining the amount of template by PCR can be performed in two ways: as relative quantitation and as absolute quantitation. Relative quantitation describes changes in the amount of a target sequence compared with its level in a related matrix. Absolute quantitation states the exact number of nucleic acid targets present in the sample in relation to a specific unit (Freeman, W, M, et al., (1999) Biotechniques, 26:112-125). Generally, relative quantitation provides sufficient information and is simpler to develop. However, when monitoring the progress of an infection, absolute quantitation is useful in order to express the results in units that are common to both scientists and clinicians and across different platforms. Absolute quantitation may also be necessary when there is a lack of sequential specimens to demonstrate changes in virus levels, no suitably standardized reference reagent or when the viral load is used to differentiate active versus persistent infection.

A very accurate approach to absolute quantitation by PCR is the use of competitive co-amplification of an internal control nucleic acid of known concentration and a wild-type target nucleic acid of unknown concentration, with the former designed or chosen to amplify with an equal efficiency to the latter (Oriaodo, C. et al., (1998) Clin. Chem. Lab. Med., 36:255-269, Becker-Andre, M. et al., (1989) Nucleic Acids Res., 17:9437-9447, Clementi, M. et al., (1995) Arch. Virol., 140:1523-1539, Gilliland, G. et al., (1990) PCR Protocols; A Guide to Methods and Applications. Academic Press, San Diego, Calif., pp. 60-69, Siebert, P. D. et al., (1992) Nature, 359:557-558). However, while conventional competitive PCR is relatively inexpensive, real-time PCR is far more convenient, reliable and better suited to quick decision-making in a clinical situation (Locatelli, G. et al., (2000). J. Clin. Microbiol., 38:4042-4048, Tanaka, N., et al., (2000) J. Med. Virol., 60:455-462). This is because conventional, quantitative, competitive PCR (qcPCR) requires significant development and optimization to ensure reproducible performance and a predetermined dynamic range for both the amplification and detection components (Ferré, F. (1992) PCR Methods Appl., 2:1-9).

Although a comparison of absolute standard curves, relative standard curves and CT values produces similar final values (Johnson, M. R. et al., (2000) Anal. Biochem., 278:175-184), the general belief remains that an internal control in combination with replicates of each sample are essential for reliable quantitation by PCR (Halford, W. P. (1999) Nat. Biotechnol, 17:835, Halford, W. P. et al., (1999) Anal. Biochem., 266:181-191). Unfortunately, real-time PCR software with the ability to calculate the concentration of an unknown by comparing signals generated by an amplified target and internal control is only beginning to emerge. This issue will hopefully be addressed in upcoming commercial releases (Kleiber, J. et al., (2000), J. Mol. Diagn., 2:158-166). Therefore, the next best approach to quantitation by PCR is the use of an external standard curve. This approach relies upon titration of an identically amplified template, in a related sample matrix, within the same experimental run. While the external standard curve is the more commonly described approach, it suffers from uncontrolled and unmonitored inter-tube variations. Because of this omission, such experiments should be described as semi-quantitative. Despite this sub-optimal approach, fluorescence data is generally collected from PCR cycles that span the linear amplification portion of the reaction where the fluorescent signal and the accumulating DNA are proportional. Because the emissions from fluorescent chemistries are temperature dependent, data is generally acquired only once per cycle at the same temperature in order to monitor amplicon yield (Wittwer, C. T. et al., (1997) Biotechniques, 22:130-138). The CT of the sample at a specific fluorescence value can then be compared with similar data collected from a series of standards by the calculation of a standard curve. The determination of the CT depends upon the sensitivity and ability of the instrument to discriminate specific fluorescence from background noise, the concentration and nature of the fluorescence-generating component and the amount of template initially present.

Real-time PCR offers significant improvements to the quantitation because of its enormous dynamic range that can accommodate at least eight logo copies of nucleic acid template (Ishiguro, T. et al., (1995) Anal. Biochem., 229:207-213, Brechtbuehl, K. et al., (2001) J. Virol. Methods, 93:105-113, Locatelli, G. et al., (2000). J. Clin. Microbiol., 38:4042-4048, Kleiber, J. et al., (2000). J. Mol. Diagn., 2:158-166, Kimura, H. et al., (1999) J. Clin. Microbiol., 37:132-136, Najioullah, F. et al., (2001) J. Virol. Methods, 92:55-64, Ryncarz, A. J. et al., (1999) J. Clin. Microbiol., 37:1941-1947, Monopoeho, S. et al., (2000) Biotechniques, 29:88-93, Alexandersen, S. et al., (2001) J. Gen. Virol., 82:747-755, Abe, A. et al., (1999) J. Clin. Microbiol., 37:2899-2903, Gruber, F. et al., (2001) Appl. Environ. Microbiol., 67:2837-2839, Moody, A. et al., (2000) J. Virol. Methods, 85:55-64). This is made possible because the data are chosen from the linear phase of amplification where conditions are optimal, rather than the end-point where the final amount of amplicon present may have been affected by inhibitors, poorly optimized reaction conditions or saturation by inhibitory PCR by-products and double-stranded amplicon. The result of taking data from the end-point is that there may not be a relationship between the initial template and final amplicon concentrations.

Real-time PCR is also an attractive alternative to conventional PCR because of its low inter-assay and intra-assay variability (Locatelli, G. et al., (2000) J. Clin. Microbiol., 38:4042-4048, Abe, A. et al., (1999) J. Clin. Microbiol., 37:2899-2903, Schutten, M. et al., (2000) J. Virol. Methods, 88:81-87) and its equivalent or greater analytical sensitivity in comparison with conventional single-round, and nested PCR (Locatelli, G. et al., (2000) J. Clin. Microbiol., 38:4042-4048, Monopoeho, S. et al., (2000) Biotechniques, 29:88-93, Kearns, A. M. et al., (2001) J. Clin. Microbiol., 3:3020-3021, Capone, R. B. et al., (2001) Clin. Cancer Res., 6:4171-4175, Leutenegger, C. M. et al., (1999) J. Virol. Methods, 78:105-116, Smith, I. L. et al., (2001) J. Virol. Methods, 9:33-40, van Elden, L. J. R. et al. (2001) J. Clin. Microbiol., 39:196-200, Lanciotti, R. S. et al., (2000) J. Clin. Microbiol., 38:4066-4071). Real-time PCR has been reported to be at least as sensitive as Southern blot (Capone, R. B. et al., (2001) Clin. Cancer Res., 6:4171-4175). However, these reports could be an over-estimate due to the choice of smaller targets, which amplify more efficiently, or due to the use of different or improved primers for the real-time assays because the use of software to design optimized primers and oligoprobes is more common.

When this increased sensitivity and broad dynamic range are combined, it is possible to quantitate template from samples containing a large range of concentrations, as is often the case in patient samples. This avoids the need for dilution of the amplicon prior to conventional detection or repeat of the assay using a diluted sample because the first test result falls outside the limits of the assay. These are problems encountered when using some conventional qcPCR assay kits, which cannot encompass high target concentrations whilst maintaining suitable sensitivity (Brechtbuehl, K. et al., (2001) J. Virol. Methods, 93:105-113, Weinberger, K. M. et al., (2000) J. Virol. Methods, 85:75-82, Schaade, L. et al., (2000) J. Clin. Microbiol, 38:4006-4009, Kawai, S. et al., (1999). J. Med. Virol., 58:121-126). The flexibility of real-time PCR is also demonstrated by its ability to detect one target in the presence of a vast excess of another target in duplexed assays (Ryncarz, A. J. et al., (1999) J. Clin. Microbiol., 37:1941-1947).

Multiplex Real-Time PCR

Multiplexing (using multiple primers to allow amplification of multiple templates within a single reaction) is a useful application of conventional PCR (Chamberlain, J., S. et al., (1988) Nucleic Acids Res., 16:11141-11156). However, its transfer to real-time PCR has confused its traditional terminology. The term multiplex real-time PCR is more commonly used to describe the use of multiple fluorogenic oligoprobes for the discrimination of multiple amplicons. The transfer of this technique has proven problematic because of the limited number of fluorophores available (Lee, L. O. et al., (1993) Nucleic Acids Res., 21:3761-3766) and the common use of a monochromatic energizing light source. Although excitation by a single wavelength produces bright emissions from a suitably selected fluorophore, this restricts the number of fluorophores that can be included (Tyagi, S. et al., (2000) Nat. Biotechnol., 18:1191-1196). Recent improvements to the design of the hairpin primers, and hairpin and nuclease oligoprobes as well as novel combinations of fluorophores such as in the bi-probe and light-up probe systems, have promised the ability to discriminate an increasing number of targets.

The discovery and application of the non-fluorescent quenchers has liberated some wavelengths that were previously occupied by the emissions from the early quenchers themselves. This breakthrough has permitted the inclusion of a greater number of spectrally discernable oligoprobes per reaction, and highlighted the need for a single non-fluorescent quencher, which can quench a broad range of emission wave-lengths (e.g. 400-600 nm). Early real-time PCR systems contained optimized filters to minimize overlap of the emission spectra from the fluorophores. Despite this, the number of fluorophores that could be combined and clearly distinguished was limited when compared with the discriminatory abilities of conventional multiplex PCR. More recent real-time PCR platforms have incorporated either multiple light-emitting diodes to span the entire visible spectrum, or a tungsten light source, which emits light over a broad range of wavelengths. When these platforms incorporate high quality optical filters it is possible to apply any current real-time PCR detection chemistries on the one machine. Nonetheless, these improvements generally allow only four-color oligoprobe multiplexing, of which one color is ideally set aside for an internal control to monitor inhibition and perhaps even act as a co-amplified competitor. Some real-time PCR designs have made use of single or multiple nucleotide changes between similar templates to allow their differentiation by TM thus avoiding the need for multiple fluorophores (Schalasta, G. et al., (2000) Infection, 28:85-91, Kearns, A. M. et al., (2001) J. Clin. Microbiol., 3:3020-3021, Espy, M. J. et al., (2000) J. Clin. Microbiol, 38:795-799, Espy, M. J. et al., (2000) J. Clin. Microbiol, 38:3116-3118, Loparev, V. N. et al., (2000) J. Clin. Microbiol., 38:4315-4319, Read, S. J. et al., (2001) J. Clin. Microbiol., 39:3056-3059, Whiley, D. M. et al., (2001) J. Clin. Microbiol., 39:4357-4361).

Future developments of novel chemistries such as combinatorial fluorescence energy transfer tags (Tong, A. K. et al., (2001) Nat. Biotechnol., 19:756-759), and improvements to the design of real-time instrumentation and software will greatly enhance the future of multiplex real-time PCR.

Signal Detection and Data Analyses

When fluorescently labeled probes are used, the fluorescence emissions at each site of a microarray may be, preferably, detected by scanning confocal laser microscopy. In one embodiment, a separate scan, using the appropriate excitation line, is carried out for each of the two fluorophores used. Alternatively, a laser may be used that allows simultaneous specimen illumination at wavelengths specific to the two fluorophores and emissions from the two fluorophores can be analyzed simultaneously (see Shalon et at, 1996, “A DNA microarray system for analyzing complex DNA samples using two-color fluorescent probe hybridization,” Genome Research 6:639-645, which is incorporated by reference in its entirety for all purposes). In a preferred embodiment, the arrays are scanned with a laser fluorescent scanner with a computer controlled X-Y stage and a microscope objective.

Sequential excitation of the two fluorophores is achieved with a multi-line, mixed gas laser and the emitted light is split by wavelength and detected with two photomultiplier tubes. Fluorescence laser scanning devices are described in Schena et al., Genome Res. 6:639-645 (1996), and in other references cited herein. Alternatively, the fiber-optic bundle described by Ferguson et al., Nature Biotech. 14: 1681-1684 (1996), may be used to monitor mRNA abundance levels at a large number of sites simultaneously.

Signals are recorded and, in a preferred embodiment, analyzed by computer, e.g., using a 12 or 16 bit analog to digital board. In one embodiment the scanned image is despeckled using a graphics program (e.g., Hijaak Graphics Suite) and then analyzed using an image gridding program that creates a spreadsheet of the average hybridization at each wavelength at each site. If necessary, an experimentally determined correction for “cross talk” (or overlap) between the channels for the two fluors may be made. For any particular hybridization site on the transcript array, a ratio of the emission of the two fluorophores can be calculated. The ratio is independent of the absolute expression level of the cognate gene, but is useful for genes whose expression is significantly modulated in association with the different responder non-responder-related condition.

Computer-Facilitated Analysis

The present invention further provides for kits comprising reagents that detect the marker sets above. In a preferred embodiment, the kit contains a microarray ready for hybridization to target polynucleotide molecules, plus software for the data analyses described above. Alternatively, the kit will comprise reagents capable of amplifying any or all of the gene markers presented in Tables 1, 3 or 4. The kit may comprise oligonucleotide pairs capable of amplifying the gene markers or subsets of those markers presented in Tables 1, 3 or 4. The subsets would contain sufficient markers to identify samples as belonging to the responder or non-responder groups. Preferred subsets would contain 2-4 or more of the markers presented in any of the Tables 1, 3 or 4. The kits may also contain enzymes to perform the amplification reactions (e.g. TAQ polymerase). In one embodiment, the kits may also contain IFN for in vitro determination of the marker gene expression phenotype (responder or non-responder).

The analytic methods described in the previous sections can be implemented by use of the following computer systems and according to the following programs and methods. A computer system comprises internal components linked to external components. The internal components of a typical computer system include a processor element interconnected with a main memory. For example, the computer system can be an Intel 8086-, 80386-, 80486-, Pentium™, or Pentium™-based processor with preferably 32 MB or more of main memory.

The external components may include mass storage. This mass storage can be one or more hard disks (which are typically packaged together with the processor and memory). Such hard disks are preferably of 1 GB or greater storage capacity. Other external components include a user interface device, which can be a monitor, together with an inputting device, which can be a “mouse”, or other graphic input devices, and/or a keyboard. A printing device can also be attached to the computer.

Typically, a computer system is also linked to network link, which can be of an Ethernet link to other local computer systems, remote computer systems, or wide area communication networks, such as the Internet. This network link allows the computer system to share data and processing tasks with other computer systems. Loaded into memory during operation of this system are several software components, which are both standard in the art and special to the instant invention. These software components collectively cause the computer system to function according to the methods of this invention. These software components are typically stored on the mass storage device. A software component comprises the operating system, which is responsible for managing computer system and its network interconnections. This operating system can be, for example, of the Microsoft Windows® family, such as Windows 3.1, Windows 95, Windows 98, Windows 2000, or Windows NT. The software component represents common languages and functions conveniently present on this system to assist programs implementing the methods specific to this invention. Many high or low level computer languages can be used to program the analytic methods of this invention. Instructions can be interpreted during run-time or compiled. Preferred languages include C/C++, FORTRAN and JAVA. Most preferably, the methods of this invention are programmed in mathematical software packages that allow symbolic entry of equations and high-level specification of processing, including some or all of the algorithms to be used, thereby freeing a user of the need to procedurally program individual equations or algorithms. Such packages include Mathlab from Mathworks (Natick, Mass.), Mathematica® from Wolfram Research (Champaign, Ill.), or S-Plus® from Math Soft (Cambridge, Mass.).

Specifically, the software component includes the analytic methods of the invention as programmed in a procedural language or symbolic package. The software to be included with the kit comprises the data analysis methods of the invention as disclosed herein. In particular, the software may include mathematical routines for marker discovery, including the calculation of correlation coefficients between clinical categories (i.e., prognosis status) and marker expression. The software may also include mathematical routines for calculating the correlation between sample marker expression and control marker expression, using array-generated, or amplification-generated fluorescence data, to determine the clinical classification of a sample.

In an exemplary implementation, to practice the methods of the present invention, a user first loads experimental data into the computer system. These data can be directly entered by the user from a monitor, keyboard, or from other computer systems linked by a network connection, or on removable storage media such as a CD-ROM, floppy disk (not illustrated), tape drive (not illustrated), ZIPS drive (not illustrated) or through the network. Next the user causes execution of expression profile analysis software that performs the methods of the present invention.

In another exemplary implementation, a user first loads experimental data and/or databases into the computer system. This data is loaded into the memory from the storage media or from a remote computer, preferably from a dynamic geneset database system, through the network. Next the user causes execution of software that performs the steps of the present invention.

Alternative computer systems and software for implementing the analytic methods of this invention will be apparent to one of skill in the art and are intended to be comprehended within the accompanying claims. In particular, the accompanying claims are intended to include the alternative program structures for implementing the methods of this invention that will be readily apparent to one of skill in the art.

### EXAMPLES

The following working examples are offered to illustrate, but not to limit the claimed invention.

Patient Characteristics

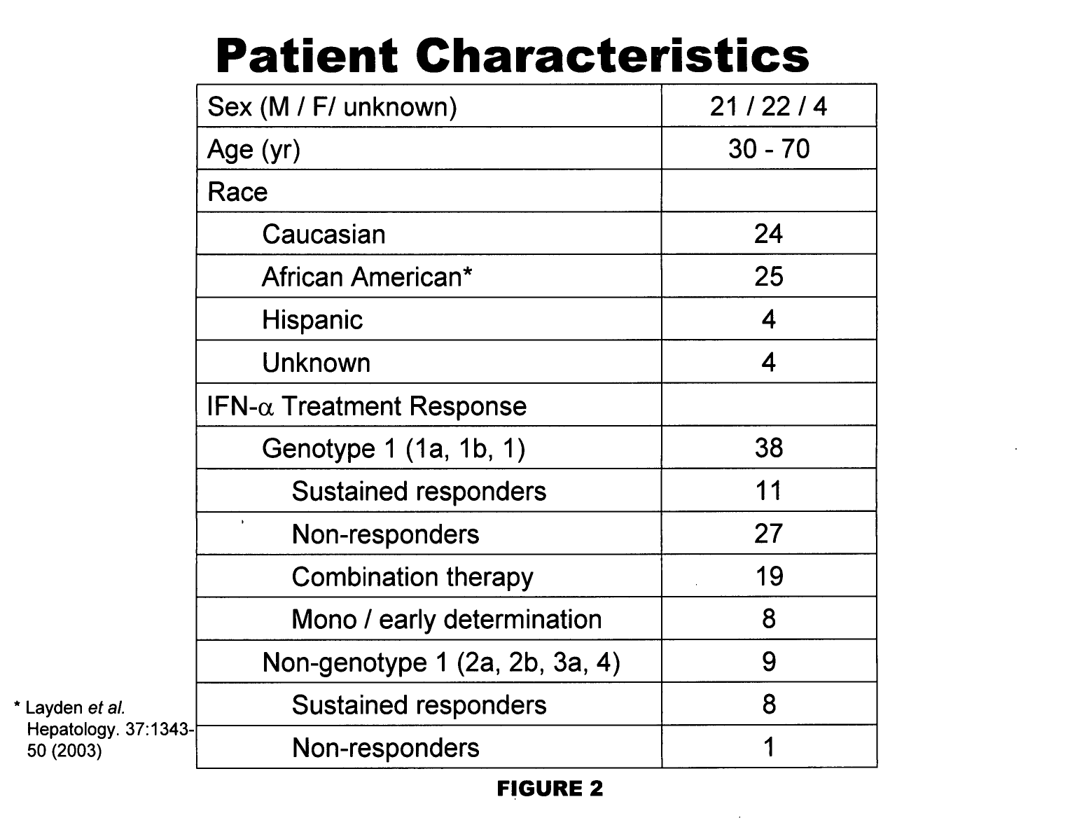

Some of the prognostic factors currently used to determine if an HCV infected patient will respond to IFN-α treatment are shown in FIG. 1. These factors include age of the patient, presence of cirrhosis/fibrosis, patient size (ie., body surface area), treatment type, viral load, and viral genotype. The odds ratios that each factor has in increasing the likelihood of responding to treatment are indicated. Host factors such as genetic polymorphisms, and cytokine levels are also indicated. The characteristics of the patients from whom samples were obtained for use in the study described below are listed in FIG. 2. Marker gene expression levels were determined in a total of 47 patients.

Sample Preparation

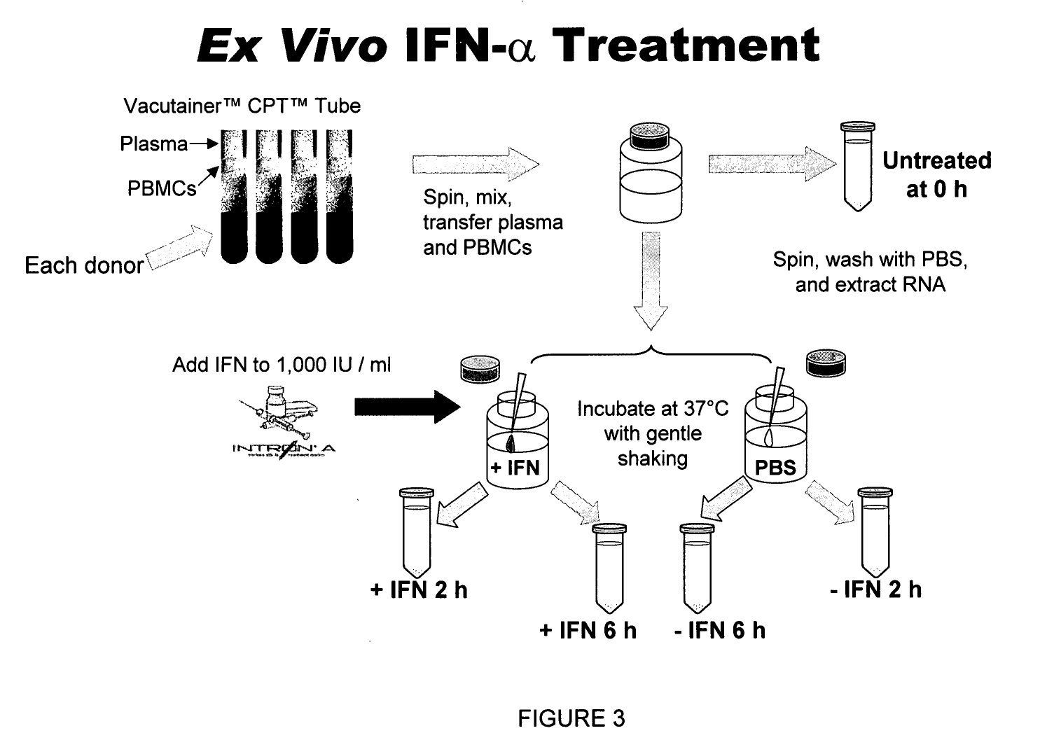

Approximately 30 mls of blood was obtained from each patient and processed as outlined in FIG. 3. Briefly, Peripheral Blood Mononuclear Cells (PBMCs) were obtained from each patient and purified. The purified PBMCs were remixed with the same plasma obtained from the patient and an aliquot was removed at 0 time and frozen. The remainder of the sample was split in half, and one half was treated with IFN-α (1,000 IU/ml, INTRON A, Schering Corp., Kenilworth, N.J.) while phosphate buffered saline was added to the other half. Both cell populations were incubated at 37° C. Half of each sample was removed at 2 hours and frozen and the remaining portion of the sample was frozen after incubation for 6 hours. After washing with phosphate buffered saline twice, total RNA was extracted from each aliquot using the RNeasy mini kit (Qiagen, Valencia, Calif.) according to manufacturer's directions. Total RNA concentration in each sample was determined with the RiboGreen® quantitation kit (Molecular Probes, Eugene, Oreg.). The expression levels of 108 marker genes (Table 3) in each sample were determined. Blood samples from seven healthy donors were also processed as above and gene expression levels for the 108 markers identified in Table 4 and the five housekeeping markers identified in Table 2 were also determined.

Kinetic RT-PCR

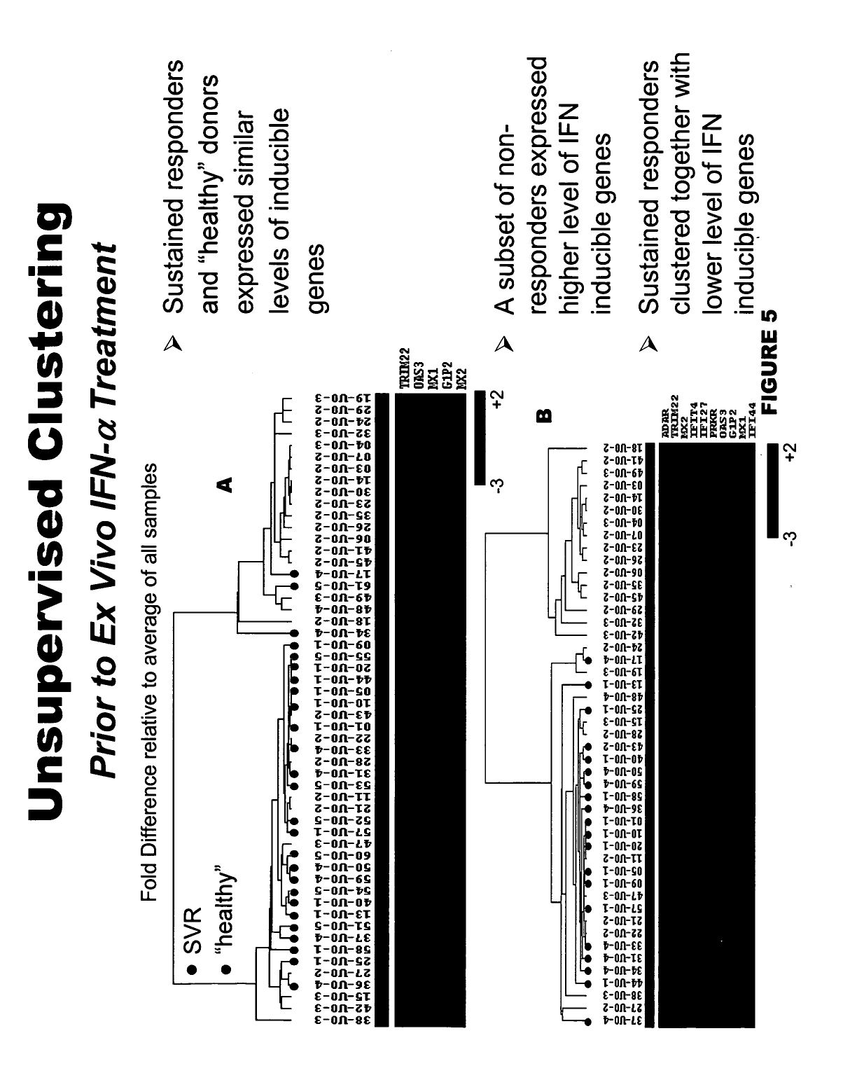

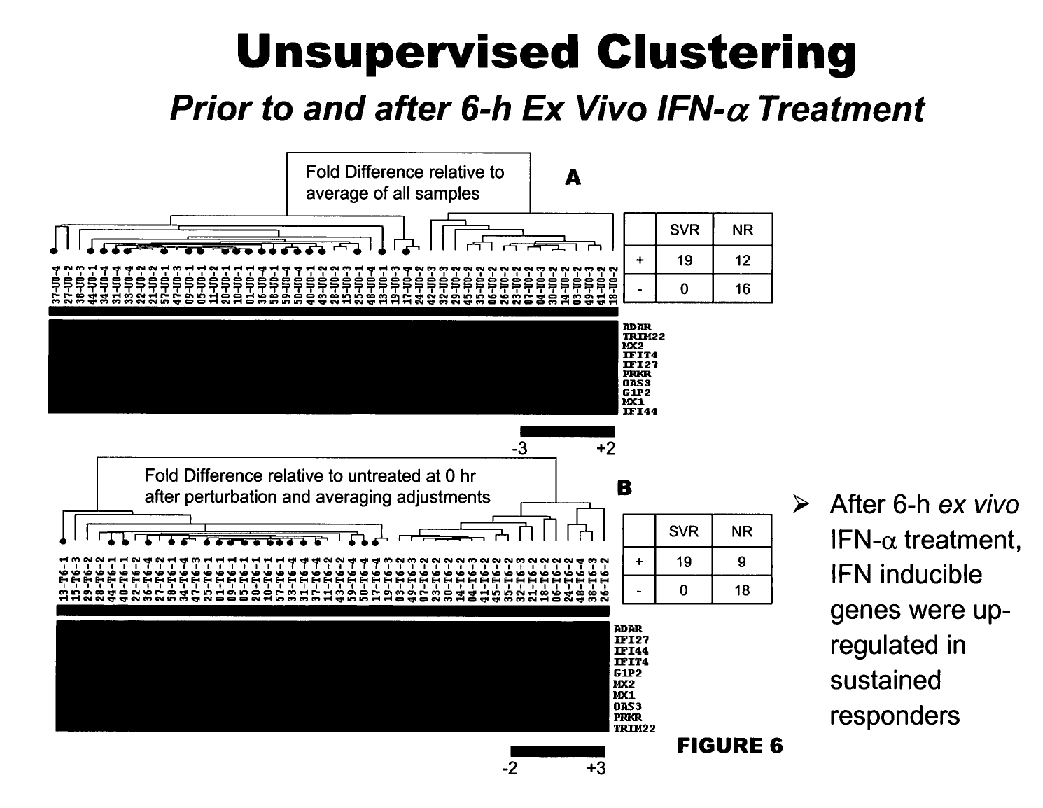

For gene expression analysis, one step RT-PCR using thermostable DNA polymerase with reverse transcription step at 60° C. was performed. 0.2 to 2.5 ng of total RNA was used in each of duplicate 15-μl reactions. The input amount of RNA was determined empirically based on the extent of amplification of a selected set of housekeeping genes present in Table 2. PCR primers for each gene in Tables 1, 3 and 4 were designed by a pipeline program. All primers flank intron(s) if possible. PCR reactions were assembled using a Biomek® FX Laboratory Workstations (Beckman Coulter Inc., Fullerton, Calif.). Each 15-μl reaction contained the following components: 50 mM Bicine, 115 mM K(OAc)₂, 8% glycerol, pH 8.0, 200 uM dATP, 200 uM dGTP, 200 uM dCTP, 400 uM dUTP, 0.2× SYBR Green, 1× ROX in 0.5% Tween-20, 0.03 uM Aptamer 46A, 3 mM Mn(OAc)₂, pH 6.5, 0.02 u/ul Uracil N-glycosylase (UNG), 0.1 u/ul rTth DNA polymerase, 200 nM each, upper and lower primer. The following PCR conditions were used: 50° C. 2 min, 95° C. 1 min, 60° C. 30 min, then 95° C. 15 sec, 60° C. 30 sec for 45 cycles, melt, 95° C. 1 min, 60° C. 1 min, ramp up to 95° C. PCR was performed using Applied Biosystems Prism® 7900HT Sequence Detection System. The expression of 5 housekeeping genes and 108 markers was profiled. The normalized copy number (HNU, Housekeeping Normalization Unit) of each transcript of interest (TOI) was determined as follows. The Standard Generating Unit (SGUs) in each sample was determined based on a “standard curve” generated from the reactions of serially diluted run off RNA transcripts. Normalization Factor was calculated based on the SGUs of five housekeeping genes (Table 2) that were expressed at relatively constant levels. The HNU of each transcript of interest was determined by dividing the SGU of each TOI by the Normalization Factor. The normalized expression level of each transcript of interest was determined for each sample and averaged across all of the samples and the color scale in FIGS. 5 and 6 represents the log difference in expression of each transcript of interest in each sample relative to the average expression across all samples. Reactions with serially diluted run-off RNA transcripts were included in each experiment to monitor inter-experimental variations.

Gene Expression Data Analysis

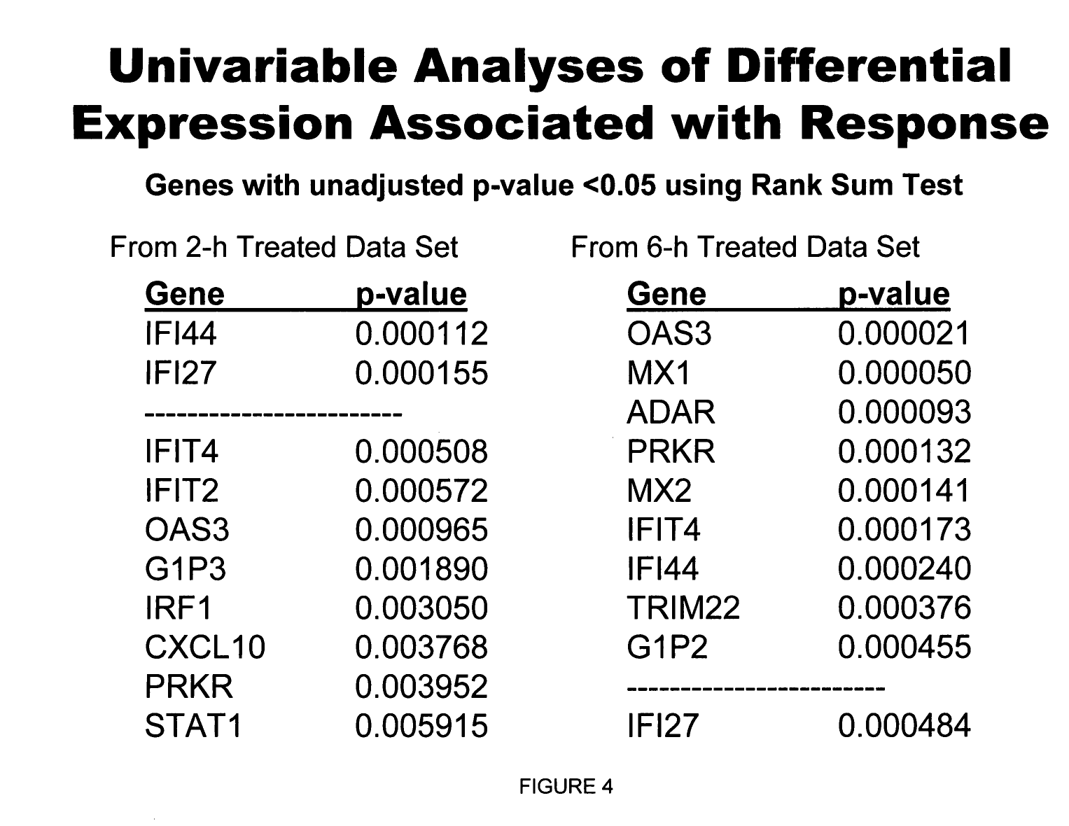

Univariable (single gene) analysis of differential expression associated with IFN response was performed. A rank sum test for differential expression between the responders and non-responders was performed. Genes that had an unadjusted p-value <0.05 using the rank sum test are presented in FIG. 4. Ten genes in the 2-hour treated data set and 6 hour treated data set showed significance. Two of the genes in the 2 hour set and nine of the genes in the 6 hour treated set survived the Bonferroni Correction. An analysis of the gene expression patterns indicates that in an unsupervised clustering of patients two groups are formed (FIG. 5), patients who exhibited sustained response to IFN treatment and healthy donors expressed similar amounts of IFN inducible genes prior to in vitro IFN treatment (FIG. 5A). The two groups were observed when a larger subset of genes were examined (FIGS. 5B and 6A). After six hours of in vitro treatment with IFN-α the same two categories were observed; however, the level of IFN inducible genes were higher in the sustained responder group relative to untreated o-hour samples than were the level of IFN inducible genes in the non-responder group (FIG. 6B). The odds ratio or positive predictive value for these results was >18 indicating that the analysis of gene expression profiles described herein is greater than any current way of predicting IFN responders in HCV infected patients (see FIG. 1).

Markers capable of differentiating between HCV infected individuals that may respond to IFN treatment and HCV infected individuals that may not respond to IFN treatment are disclosed. The expression profiles of these markers may be determined from samples obtained from HCV infected patients using in vitro IFN treatment of the samples as described above, prior to the onset of treatment of the individual or from samples obtained shortly after treatment of the individual has begun.

### REFERENCES CITED

All publications and patents cited in this specification are herein incorporated by reference in their entirety. Various modifications and variations of the described compositions, methods and systems of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the above-described modes for carrying out the invention that are obvious to those skilled in the field of molecular biology, genetics and related fields are intended to be within the scope of the following claims.

1. A method of selecting an HCV infected individual for treatment with IFN-α, comprising:
(a) exposing cells obtained from the individual to IFN-α in vitro; and
(b) detecting the altered expression of at least 3 markers as listed in Table 4, as compared to cells obtained from the individual prior to IFN-α exposure;
wherein altered expression of said markers is correlated with a positive response to IFN-α treatment of the HCV infection.

2. The method of claim 1 wherein the cells are exposed to IFN-α in vitro for 1-20 hours.

3. The method of claim 1, wherein the cells are peripheral blood mononuclear cells.

4. The method of claim 1, wherein the markers whose expression is altered are ADAR, IFI27, IFI44, OAS3, MX1, MX2, PRKR, IFIT4, TRIM22, and G1P2.

5. The method of claim 1, wherein the cells are obtained from the individual prior to any in vivo IFN-α administration in said individual.

6. The method of claim 1, wherein the level of expression of the marker is altered at least 2 fold relative to their expression levels in cells obtained prior to their exposure to IFN-α in vitro.

7. A method of identifying an HCV infected individual for continued treatment with IFN-α, comprising:
(a) obtaining cells from an HCV infected individual who has received in vivo administration of IFN-α; and
(b) detecting the altered expression of at least 3 markers as listed in Table 4, as compared to cells obtained from the individual prior to IFN-α exposure;
wherein altered expression of said markers is correlated with a positive response to IFN-α treatment of the HCV infection.

8. The method of claim 7, wherein the cells are obtained from the individual 2-72 hours after in vivo administration of IFN-α

9. The method of claim 7, wherein the cells are peripheral blood mononuclear cells.

10. The method of claim 7, wherein the markers whose expression is altered are ADAR, IFI27, IF144, OAS3, MX1, MX2, PRKR, IFIT4, TRIM22, and G1P2.

11. The method of claim 7, wherein the level of expression of the marker is altered at least 2 fold relative to their expression levels in cells obtained prior to their exposure to IFN-α in vivo.

12. A method of treating an HCV-infected individual, comprising administering a therapeutically effective amount of IFN-α in said individual, whose cells have been shown in an in vitro assay to exhibit altered expression of at least 3 markers as listed in Table 4.

13. The method of claim 12, wherein the cells are peripheral blood mononuclear cells.

14. The method of claim 12, wherein the markers whose expression is altered are ADAR, IFI27, IF144, OAS3, MX1, MX2, PRKR, IFIT4, TRIM22, and G1P2.

15. The method of claim 12, wherein the level of expression of the marker is altered at least 2 fold relative to their expression levels in cells obtained prior to their exposure to IFN-α.

16. A method of identifying an HCV infected individual for discharge from treatment with IFN-α, comprising:
(b) exposing cells obtained from an HCV infected individual with IFN-α in vitro; and
(b) detecting a lack of altered expression of at least 3 markers as listed in Table 4, as compared to cells obtained from the individual prior to IFN-α exposure;
wherein altered expression of said markers is correlated with a positive response to IFN-α treatment of the HCV infection.

17. The method of claim 16, wherein the level of expression of the markers is altered 1.5 fold or less relative to their expression levels in cells obtained prior to their exposure to IFN-α in vitro.

18. A kit comprising reagents for detecting the expression of at least 3 markers as listed in Table 4.

19. The kit of claim 18, wherein the reagents further comprise amplification primers.

20. The kit of claim 18, wherein the reagents further comprise hybridization probes.
